(19) United States
(12) Patent Application Publication
(10) Pub. No.: US 2012/0115955 A1
(43) Pub. Date: May 10, 2012

(54) Methods for tumor treatment and adipogenesis differentiation

(75) Inventors: Carlos Simon, Valencia (ES); Cristobal Aguilar-Gallardo, Valencia (ES)

(73) Assignee: Centro de Investigacion Principe Felipe, Valencia (ES)

(21) Appl. No.: 13/251,529

(22) Filed: Oct. 3, 2011

Related U.S. Application Data
(60) Provisional application No. 61/389,358, filed on Oct. 4, 2010.

(51) Int. Cl. A61K 31/201; C12N 5/071; A61P 35/00

(52) U.S. Cl. 514/560; 435/377

(57) ABSTRACT

The present invention provides methods and pharmaceutical compositions for treating tumors.

### CROSS REFERENCE

This application claims priority to U.S. Provisional Patent Application Ser. No. 61/389,358 filed Oct. 4, 2010, incorporated by reference herein in its entirety.

### BACKGROUND

Recent studies have described the transdifferentiation of committed cells to a different cell lineage through gene overexpression or small molecule contact, and the possible use of transdifferentiation as a therapeutic strategy. However, there is currently no treatment based on cell differentiation therapy for human solid tumors.

### SUMMARY OF THE INVENTION

In a first aspect, the present invention provides methods for treating a tumor, comprising administering to a subject with a tumor an amount effective of a formulation comprising one or more fatty acids selected from the group consisting of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof, to treat the tumor. In one embodiment, the one or more fatty acids consist of palmitoleic acid, oleic acid, and linoleic acid. In another embodiment, the one or more fatty acids consist of palmitoleic acid, oleic acid, and petroselenic acid. In a further preferred embodiment, the tumor is a solid tumor.

In a second aspect, the present invention provides methods for inducing adipogenesis in an adult non-adipogenic cell, comprising contacting the adult non-adipogenic cell with an amount effective of a formulation comprising one or more fatty acids selected from the group consisting of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof, to induce adipogenesis in the adult non-adipogenic cell. In one embodiment, the one or more fatty acids consist of palmitoleic acid, oleic acid, and linoleic acid. In another embodiment, the one or more fatty acids consist of palmitoleic acid, oleic acid, and petroselenic acid.

In a third aspect, the present invention provides pharmaceutical compositions comprising

(a) a fatty acid cocktail, wherein fatty acids in the fatty acid cocktail consist of two or more of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof; and

(b) a pharmaceutically accepted carrier.

In one embodiment, the fatty acids in the fatty acid cocktail are palmitoleic acid, oleic acid, and linoleic acid. In another embodiment, the fatty acids in the fatty acid cocktail are palmitoleic acid, oleic acid, and petroselenic acid.

### BRIEF DESCRIPTION OF THE FIGURES

FIG. 1. KOSR induces modifications in the FSC and SSC parameters of HCCLs. (A). FSC and SSC plots from the HCCLs cultured with conditioned media. MCF-7 and MALME-3M cells were grown on 6-well plates (10⁵ cells/well) and cultured in HES+20% KOSR conditioned with VAL9 for 48 hours. As a negative control, HES+20% KOSR was conditioned with the feeder only, and in the absence of VAL9. Cancer cells were also cultured in RPMI+10% FBS (considered time=0). Quantification in the FSC and SSC parameters was assessed by flow cytometry and further determined using the FlowJo software. Fold induction indicates the ratio between treated conditions and the untreated condition (time=0). (B). FSC and SSC plots from the HCCLs cultured with KOSR. MALME-3M, MCF-7, SK-OV-3, and HUH-7 cells were cultured with HES+20% KOSR for 24 hours at a concentration of 10⁵ cells/well in 6-well plates. As controls, cancer cells were cultured in RPMI-1640 (considered time=0) or in a HES medium containing 10% of FBS. Quantification in the FSC and SSC parameters was assessed by the FlowJo software. Fold induction was estimated by calculating the ratio between treated conditions and the untreated condition (time=0).

FIG. 2. Electron Microscopy of LDs biogenesis in the HCCLs cultured with KOSR. (A-H). Electron microscopy (EM) of HCCLs: MALME-3M (A-D), MCF-7 (E-F) and HUH-7(G-H) was performed as indicated in the Materials and Methods section. Cells were grown in the presence of HES+20% KOSR for 48 hours (B, C, D, F and H). As a negative control, MALME-3M (A), MCF-7 (E) and HUH7 (G) cells were grown with RPMI-10% FBS.

FIG. 3. Oil red and Nile red quantifications of neutral lipids induced by KOSR. (A-O). Oil-red staining of HCCLs cultured with KOSR. HCCLs: MALME-3M (A-C), SK-OV-3 (D-F), and MCF-7(G-I) were cultured as described in FIG. 1B. Oil-red and hematoxylin stainings were performed as described in the Materials and Methods section. As a negative control, KOSR was replaced with 10% of FBS (C, F, I, L, O). VAL9 was grown on HES+20% KOSR and considered a negative control (N). The preadipocyte 3T3-L1 cell line was grown under the same conditions as HCCLs, and was considered a positive control for LDs (K). Magnification was ×40. (P). HCCLs (MALME-3M, MCF-7, SK-OV-3) were cultured as described in FIG. 1B, and nile-RED staining was performed as described in the Materials and Methods section. Quantification was assessed by the FlowJo software. Fold induction was estimated by calculating the ratio between treated conditions and the untreated condition (control).

FIG. 4. PPARG up-regulation in MCF-7 cells after KOSR treatment. RT-PCR analysis of MCF-7 cultured with KOSR. Gene expression analysis for PPARG1, ADIPOQ, LPL, MLYCD, MPZL2 and GAPDH (loading control). MCF-7 cells were cultured with (w) and without (w/o) 20% of KOSR for 48 hours. The expressions of these genes were also analyzed in VAL9 (negative control) and white adipose tissues (WAT, positive control, +). (−) denotes RT-PCR w/o RNA and in the presence of H₂O. PPARG1 was used as a positive control of the KOSR-induced lipogenesis in each gene testing. The results are representative of at least three independent experiments for each gene.

FIG. 5. LDs accumulation induced by albuMAX in MALME-3M cells. (A). FSC and SSC plots from the HCCLs cultured with albuMAX. The MALME-3M cells cultured on 6-well plates (10⁵ cells/well) were cultured for 48 hours w and w/o 1.6% of albuMAX. Cells were also cultured with RPMI-10% FBS and considered negative controls. Quantification in the FSC and SSC parameters was assessed by flow cytometry and further analyzed by the FlowJo software. Fold induction indicates the ratio between treated conditions and the untreated condition (control). (B-D). The MALME-3M cells cultured on 6-well plates were cultured for 48 hours w (D) and w/o (C) 1.6% of albuMAX. Controls (time=0) were considered when cells were cultured with RPMI+10% FBS (B). Pictures showing Oil-RED staining are provided. Magnification was ×40. (E-H). Confocal microscopy analysis of the Nile-Red stained-MALME-3M cells cultured either in the presence (F, H) or absence (E, G) of 1.6% of albuMAX. The cells fixed with 4% of paraformaldehyde were stained with Nile-RED, as indicated in the Materials and Methods (E, F). Next cells were mounted with Prolong antifade containing DAPI for nuclear staining (G, H). Images (×63) are shown. (I). MALME-3M cells were cultured as described in FIG. 1B and nile-RED staining was performed as described in the Materials and Methods section. Quantification was assessed by the FlowJo software. Fold induction was estimated by calculating the ratio between treated conditions and the untreated condition (control).

FIG. 6. Oleic, linoleic, palmitoleic and petroselinic acids, but not albumin, are the transdifferentiating lipids of HCCLs to an adipocyte-like phenotype. MALME-3M cells were cultured in the presence of 10 μg/ml of docosahexaenoic acid, 100 μg/ml of nervonic acid, 100 μg/ml of erucic acid, 10 μg/ml of arachidonic acid, 100 μg/ml of linolenic acid, 100 μg/ml of linoleic acid, 100 μg/ml of oleic acid, 100 μg/ml of palmitoleic acid, 100 μg/ml of elaidic acid, or 100 μg/ml petroselinic acid for 12 hours. Fatty acids were dissolved in DMSO; therefore the cells cultured with RPMI+10% FBS and treated with DMSO only were considered negative controls. The Nile-Red analysis was performed as described in the Material and Methods section. Quantification in the FL2 parameter was assessed by the FlowJo software. Fold induction was estimated by calculating the ratio between treated conditions and the untreated condition (control). The results are representative of three independent experiments.

FIG. 7. Electron Microscopy of LDs biogenesis in both the HUH-7 and MALME-3M induced by the POT and POL fatty acids cocktails. Cells were cultured in the presence of the indicated fatty acids cocktails at a concentration of 100 μg/ml for 48 hours (B-D, F-H). As a negative control, HUH-7 and MALME-3M were grown with RPMI-10% FBS (A, E). Bar scales: 10, 5 and 1 μm. The presence of massive multilocular (in MALME-3M) or unilocular (in HUH-7) LDs can be observed with LDs in close proximity to mitochondria and the rough endoplasmic reticulum, even with a polarization pattern that further demonstrates the specificity of this effect.

FIG. 8. AlbuMAX, a component of the KOSR, induces cancer terminal transdifferentiation and tumour reduction in vivo. MCF-7 cancer cells were transdifferentiated towards adipocyte-like cells through incubation with HES media during 14 days, these cells were termed Adipo-MCF-7 cells. In parallel, we grew MCF-7 cancer cells in the presence of RPMI-(10% FBS) used as negative control. Next, we subcutaneously transplanted 10⁵ human cancer cells from each cell type into male NOD/SCID mice for in vivo analysis of tumor progression. After 2 months of transplantation, analysis of tumour cell size in transplanted NOD/SCID mice indicated a profound reduction in tumour growth induced by Adipo-MCF-7 cells compared with their counterpart MCF-7 cells. Notably, H&E analysis in tumours originated from Adipo-MCF-7 cells exhibited features of adipocyte terminal transdifferentiation as evaluated by the presence of condensed nucleus excluded from the cytoplasm and the presence of large lipid droplets resembling to white adipocyte tissues (WAT). Thus, these results indicate that Adipo-MCF-7 cells exhibit an increase potential in adipocyte-like features in vivo, such as the acquisition of hallmarks of terminal differentiation, which are responsible of less tumorigenicity.

### DETAILED DESCRIPTION OF THE INVENTION

In a first aspect, the present invention provides methods for treating a tumor, comprising administering to a subject with a tumor an amount effective of a formulation comprising one or more fatty acids selected from the group consisting of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof, to treat the tumor. In a preferred embodiment, the method comprises administering two or more of the fatty acids selected from the group.

The inventors have shown in the examples that the use of the recited fatty acids serves to transform tumor cells to a less aggressive cellular phenotype such as an adipocyte-like cell, thus indicating the value of the methods for treating tumors.

In one preferred embodiment, the one or more fatty acids comprise or consist of palmitoleic acid, oleic acid, and linoleic acid (“POL” cocktail). In another preferred embodiment, the one or more fatty acids comprise or consist of palmitoleic acid, oleic acid, and petroselenic acid (“POT” cocktail). As shown in the examples that follow, these formulations provide for improved efficiency in blocking cancer progression. In a preferred embodiment, the fatty acids in the formulation consist of one or more of the recited fatty acids. In this embodiment, the formulation may comprise other components useful for treating tumors or for preparing the formulation, including but not limited to those discussed below. In one embodiment that can be combined with any embodiment herein, the formulation further comprises a carrier protein, including but not limited to albumin, wherein the carrier protein serves to form a lipid protein complex with fatty acids in the formulation. This embodiment improves internalization of the fatty acids into the cells and improves the therapeutic benefit of the formulation accordingly. In one embodiment, the carrier protein may comprise anywhere between 1-70% of the formulation, preferably between 20-70%.

The methods can be used to treat any suitable tumor type. In one preferred embodiment, the tumor is a solid tumor, such as a sarcoma, carcinoma, or lymphoma. In another preferred embodiment, the tumor comprises a tumor selected from the group consisting of melanoma, hepatic carcinoma, breast carcinoma, ovarian carcinoma, and cancer stem cells.

In one preferred embodiment, the tumor comprises a hepatocarcinoma and the one or more fatty acids used comprise or consist of petroselenic, oleic and palmitoleic acid. In another preferred embodiment the tumor comprises a melanoma, and the one or more fatty acids comprise or consist of linoleic, oleic and palmitoleic acid.

As used herein, the phrase “an amount effective” refers to the amount of inhibitor that provides a suitable treatment effect.

As used herein, “treating tumors” means accomplishing one or more of the following: (a) reducing tumor mass; (b) slowing the increase in tumor mass; (c) reducing tumor metastases; (d) slowing the incidence of tumor metastases; (e) limiting or preventing development of symptoms characteristic of cancer; (f) inhibiting worsening of symptoms characteristic of cancer; (g) limiting or preventing recurrence of symptoms characteristic of cancer in subjects that were previously symptomatic; (i) increasing subject survival time; and (j) limiting or reducing morbidity of therapy by enhancing current therapies, permitting decreased dose of current standard of care therapies. As used herein, the phrase “pharmaceutically acceptable salt” refers to both pharmaceutically acceptable acid and base addition salts and solvates. Such pharmaceutically acceptable salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

The subject can be any mammal, preferably a human.

While not being bound by any specific mechanism of action, the inventors believe that the methods of the invention work by causing transdifferentiate of tumor cells in the tumor into adipocyte-like cells. Thus, in another preferred embodiment that can be combined with any of the other embodiments herein, the one or more fatty acids serve to transdifferentiate tumor cells in the tumor into adipocyte-like cells. As will be understood by those of skill in the art, this embodiment does not require that all tumor cells are transdifferentiated into adipocyte-like cells, but only that a sufficient number of tumor cells are transdifferentiated to treat the tumor. As used herein, “transdifferentiation” means that the tumor cell differentiates into a different type of cell. As used herein, an “adipocyte-like cell” is a former tumor cell in which biogenesis of lipid droplets (LDs) can be detected and up-regulation of the adipogenic master regulator PPARG can be detected.

As used herein, “up-regulate” means at least a 10% induction in expression and/or activity of PPARG compared to control (such as tumor cells not treated with the formulation); preferably at least a 20%, 30%, 40%, 50%, 60%, 70%, 80%, 90%, or greater induction in expression and/or activity.

The actual fatty acid dosage range for administration is based on a variety of factors, including the age, weight, sex, medical condition of the individual, the severity of the condition, and the route of administration. An effective amount of the one or more fatty acids that can be employed ranges generally between 0.01 μg/kg body weight and 10 mg/kg body weight, preferably ranging between 0.05 μg/kg and 5 mg/kg body weight, more preferably between 1 μg/kg and 5 mg/kg body weight, and even more preferably between about 10 μg/kg and 5 mg/kg body weight.

When combinations of fatty acids are used in the formulations, any ratio of such fatty aids suitable for a given treatment regimen can be used. In one embodiment, each fatty acid in the formulation is present in an approximately similar amount. In another embodiment, the ratio of one fatty acid to any other fatty acid in the formulation ranges between 10:1 and 1:10; in various further embodiments, between 9:1 and 1:9; 8:1 and 1:8; 7:1 and 1:7; 6:1 and 1:6; 5:1 and 1:5; 4:1 and 1:4; 3:1 and 1:3; 2:1 and 1:2; and 1:1. In a further embodiment, the methods of the invention further comprise treating the subject with surgery (to remove the tumor), chemotherapy and/or radiation therapy.

The methods of the invention may permit a reduction or elimination of the chemotherapy and/or radiation dosage necessary to inhibit tumor growth and/or metastasis.

As used herein, “radiotherapy” includes but is not limited to the use of radio-labeled compounds targeting tumor cells. Any reduction in chemotherapeutic or radiation dosage benefits the patient by resulting in fewer and decreased side effects relative to standard chemotherapy and/or radiation therapy treatment. In this embodiment, the formulation may be administered prior to, at the time of, or shortly after a given round of treatment with chemotherapeutic and/or radiation therapy. In a preferred embodiment, the formulation is administered prior to or simultaneously with a given round of chemotherapy and/or radiation therapy. In a most preferred embodiment, the formulation is administered prior to or simultaneously with each round of chemotherapy and/or radiation therapy. The exact timing of compound administration will be determined by an attending physician based on a number of factors, but the formulation is generally administered between 24 hours before a given round of chemotherapy and/or radiation therapy and simultaneously with a given round of chemotherapy and/or radiation therapy. The methods of the invention are appropriate for use with chemotherapy using one or more cytotoxic agent (ie: chemotherapeutic), including, but not limited to, cyclophosphamide, taxol, 5-fluorouracil, adriamycin, cisplatinum, methotrexate, cytosine arabinoside, mitomycin C, prednisone, vindesine, carbaplatinum, and vincristine. The cytotoxic agent can also be an antiviral compound which is capable of destroying proliferating cells. For a general discussion of cytotoxic agents used in chemotherapy, see Sathe, M. et al., Cancer Chemotherapeutic Agents: Handbook of Clinical Data (1978), hereby incorporated by reference. When administered as a combination, the therapeutic agents can be formulated as separate compositions that are given at the same time or different times, or the therapeutic agents can be given as a single composition.

In a second aspect, the present invention provides methods for inducing adipogenic differentiation in a non-adipogenic cell, comprising contacting the non-adipogenic cell with an amount effective of a formulation comprising one or more fatty acids selected from the group consisting of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof, to induce adipogenesis in the non-adipogenic cell.

The inventors have discovered that the methods of this second aspect of the invention can be used to induce adipogenic differentiation in non-adipogenic cell in 24 hours, a huge improvement over existing methods and kits that require 15-30 days to induce adipogenic differentiation.

All embodiments of the first aspect of the invention can be used in this second aspect of the invention, and all common terms have the same meaning. All embodiments of the formulation disclosed for the first aspect are also preferred for use in this second aspect. For example, inducing adipogenesis means to transdifferentiate the adult non-adipogenic cell into an “adipocyte-like cell”as defined in the first aspect of the invention.

As used herein, an “non-adipogenic cell” means that the cells (prior to contacting with the formulation) comprise or consist of cells that are not “adipocyte-like cells” as defined in the first aspect of the invention. Any suitable “non-adipogenic cell” may be contacted as desired for a given purpose, including but not limited to mesenchymal stem cells, human adipose-derived stem cells, embryonic stem cells, and non-stem cells of various types.

The methods of this second aspect of the invention can be carried out in vitro or in.vivo. When used in vitro, any suitable culture conditions can be used as appropriate for the specific cell type used. Standard growth media and supplemented growth factors can be used as appropriate for a given cell type; non-limiting examples are provided in the examples below both for adult cells and embyronic stem cells. Alterations in gene expression and lipid droplet accumulation resulting from the methods can be assessed using any suitable means, including but not limited to those described in the examples that follow.

When used in vivo, the subject being treated may be any mammal, preferably a human. The actual fatty acid dosage range for in vivo administration is based on a variety of factors, including the age, weight, sex, medical condition of the individual, the severity of the condition, and the route of administration. An effective amount of the one or more fatty acids that can be employed ranges generally between 0.01 μg/kg body weight and 10 mg/kg body weight, preferably ranging between 0.05 μg/kg and 5 mg/kg body weight, more preferably between 1 μg/kg and 5 mg/kg body weight, and even more preferably between about 10 μg/kg and 5 mg/kg body weight.

When combinations of fatty acids are used in the formulations, any ratio of such fatty acids suitable for a given treatment regimen can be used. In one embodiment, each fatty acid in the formulation is present in an approximately similar amount. In another embodiment, the ratio of one fatty acid to any other fatty acid in the formulation ranges between 10:1 and 1:10; in various further embodiments, between 9:1 and 1:9; 8:1 and 1:8; 7:1 and 1:7; 6:1 and 1:6; 5:1 and 1:5; 4:1 and 1:4; 3:1 and 1:3; 2:1 and 1:2; and 1:1. In a further embodiment, the methods of the invention further comprise treating the subject with surgery (to remove the tumor), chemotherapy and/or radiation therapy.

In one embodiment that can be combined with any embodiment herein, the formulation further comprises a carrier protein, including but not limited to albumin, wherein the carrier protein serves to form a lipid protein complex with fatty acids in the formulation. This embodiment improves internalization of the fatty acids into the cells and improves the therapeutic benefit of the formulation accordingly. In one embodiment, the carrier protein may comprise anywhere between 1-70% of the formulation, preferably between 20-70%.

Pharmaceutically acceptable salts in accordance with the present invention are the salts with physiologically acceptable bases and/or acids well known to those skilled in the art of pharmaceutical technique. Suitable salts with physiologically acceptable bases include, for example, alkali metal and alkaline earth metal salts, such as sodium, potassium, calcium and magnesium salts, and ammonium salts and salts with suitable organic bases, such as methylamine, dimethylamine, trimethylamine, piperidine, morpholine and triethanolamine. Suitable salts with physiologically acceptable acids include, for example, salts with inorganic acids such as hydrohalides (especially hydrochlorides or hydrobromides), sulphates and phosphates, and salts with organic acids. The pharmaceutical compositions of the invention may include admixtures of the fatty acids, or pharmaceutically acceptable salt thereof, and the one or more other compounds, as well as separate unit dosages of each that are manufactured for combinatorial use. Such separate unit dosages may be administered concurrently or sequentially as determined by the clinician.

In all aspects formulation comprises one or more pharmaceutically acceptable carriers appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the formulations may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In a preferred embodiment of each of the above aspects of the invention, the formulations are prepared for oral administration. As such, the formulation can be in the form of, for example, a tablet, a hard or soft capsule, a lozenge, a cachet, a dispensable powder, granules, a suspension, an elixir, a liquid, or any other form reasonably adapted for oral administration. The formulation can further comprise, for example, buffering agents. Tablets, pills and the like additionally can be prepared with enteric coatings. Unit dosage tablets or capsules are preferred.

Formulations suitable for buccal administration include, for example, lozenges comprising the recited fatty acids, or pharmaceutically acceptable salts thereof and a flavored base, such as sucrose, acacia tragacanth, gelatin, and/or glycerin.

Liquid dosage forms for oral administration can comprise pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents.

These formulations can be prepared by any suitable method that includes the step of bringing into association the recited fatty acids, or pharmaceutically acceptable salts thereof, and the pharmaceutically acceptable carrier.

In a third aspect, the present invention provides pharmaceutical compositions comprising

(a) a fatty acid cocktail, wherein fatty acids in the fatty acid cocktail consist of two or more of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof; and

(b) a pharmaceutically accepted carrier.

The compositions can be used, for example, in the methods disclosed in the first and second aspects above.

All embodiments of the first and second aspects of the invention can be used in this third aspect of the invention, and all common terms have the same meaning

In one preferred embodiment, the fatty acids in the fatty acid cocktail are palmitoleic acid, oleic acid, and linoleic acid. In another preferred embodiment, the fatty acids in the fatty acid cocktail are palmitoleic acid, oleic acid, and petroselenic acid.

Suitable dosage units for the fatty acids of this third aspect of the invention are based on a variety of factors, including the age, weight, sex, medical condition of the individual, the severity of the condition, and the route of administration. Exemplary dosage units generally are those that can provide between 0.01 μg/kg body weight and 10 mg/kg body weight to a subject in need thereof, preferably ranging between 0.05 μg/kg and 5 mg/kg body weight, more preferably between 1 μg/kg and 5 mg/kg body weight, and even more preferably between about 10 μg/kg and 5 mg/kg body weight. The dosage units may be administered at any suitable interval as deemed appropriate by an attending physician, such as once or more per day.

In one embodiment, each fatty acid in the composition is present in an approximately similar amount. In another embodiment, the ratio of one fatty acid to any other fatty acid in the formulation ranges between 10:1 and 1:10; in various further embodiments, between 9:1 and 1:9; 8:1 and 1:8; 7:1 and 1:7; 6:1 and 1:6; 5:1 and 1:5; 4:1 and 1:4; 3:1 and 1:3; 2:1 and 1:2; and 1:1.

In one embodiment that can be combined with any embodiment herein, the formulation further comprises a carrier protein, including but not limited to albumin, wherein the carrier protein serves to form a lipid protein complex with fatty acids in the formulation. This embodiment improves internalization of the fatty acids into the cells and improves the therapeutic benefit of the formulation accordingly. In one embodiment, the carrier protein may comprise anywhere between 1-70% of the formulation, preferably between 20-70%.

In a further embodiment, the pharmaceutical compositions further comprise one or more further therapeutics for treating a tumor. In one embodiment, the further therapeutic comprises a cytotoxic agent, including but not limited to cyclophosphamide, taxol, 5-fluorouracil, adriamycin, cisplatinum, methotrexate, cytosine arabinoside, mitomycin C, prednisone, vindesine, carbaplatinum, vincristine, or an antiviral compound.

Pharmaceutically acceptable salts and pharmaceutically acceptable carriers in this third aspect of the invention are as described above in the first and second aspects.

In a preferred embodiment, the pharmaceutical composition comprises an oral dosage form, including but not limited to a tablet, a hard or soft capsule, a lozenge, a cachet, a dispensable powder, granules, a suspension, an elixir, a liquid, or any other form reasonably adapted for oral administration.

All embodiments of the invention can be combined with other embodiments unless the context clearly dictates otherwise.

### EXAMPLE 1

### Summary

Differentiation therapy pursues the discovery of novel molecules to transform cancer progression to less aggressive phenotypes by mechanisms involving enforced cell transdifferentiation. Here, we have integrated cell culture techniques with electron and confocal microscopy, flow cytometry and RT-PCR approaches for the identification of transdifferentiating adipogenic programs in human cancer cell lines (HCCLs). A mini-screen of unsatturated fatty acids associated with albumin (ALB) reveals that palmitoleic, oleic and linoleic acids trigger remarkable modifications in the forward and side scatter parameters of hepatocarcinoma HUH-7, ovarian carcinoma SK-OV-3, breast adenocarcinoma MCF-7 and melanoma MALME-3M. Concominantly with this process, massive biogenesis of lipid droplets (LDs) and up-regulation of the adipogenic master regulator PPARG were detected, resulting in the transdifferentiation of the indicated HCCLs into adipocyte-like cells. These findings suggest the possibility of switching the original identity of HCCLs through unsaturated fatty acid-induced transdifferentiation toward an adipogenic phenotype as a novel strategy in cancer differentiation therapy.

### Experimental Procedures

### Human Cancer Cell Lines (HCCLs)

MALME-3M melanoma (ATTC# HTB-64), HUH-7 hepatocarcinoma, MCF-7 breast adenocarcinoma (ATTC# HTB-22) and SK-OV-3 ovarian adenocarcinoma (ATTC# HTB-77). HCCLs were split into growth media containing RPMI-1640, 10% fetal bovine serum (FBS) and 2 mM glutamine following a standard 3T3 protocol (Todaro and Green, 1963).

### Human Embryonic Stem Cells

VAL9 [http://www.isciii.es/htdocs/terapia/pdf/Documento_Deposito_VAL_{—}9.pdf] was split onto a monolayer of irradiated human foreskin fibroblasts (FSK) (ATTC# CRL-2429). hESCs were cultured at a density of 10⁴ cells/cm² in HES growth media (KO-DMEM, 20% knock-out serum replacement-KOSR, 2 mM glutamine, 1% non-essential amino acids, 50 μM β-mercaptoethanol and 10 ng/ml human recombinant bFGF). VAL9 colonies were manually expanded using Pasteur pipettes tips every 4 days. Briefly, 40 hESC colony fragments (containing 10,000 cells/fragment) were split into new plates (6-well plates), and growth media were replaced daily.

### Electron Microscopy

HCCLs were seeded on Permanox slides (Nalge Nunc International, Naperville, Ill.) at a density of 2000 cells/well. Cells were fixed with 3.5% glutaraldehyde (1 h, 37° C.), treated with 2% osmium tetroxide (1 h, RT) and stained with 2% uranyl acetate away from light (2 h, 4° C.). Cells were rinsed in sodium phosphate buffer (0.1 M), dehydrated in ethanol, and infiltrated overnight in araldite resins (Durcupan, Fluka, Buchs SG, Switzerland). After polymerization, embedded cultures were detached from the chamber slide and glued to Araldite blocks. Semi-thin sections (1.5 μm) were cut with an Ultracut UC-6 (Leica, Heidelberg, Germany), mounted onto slides and stained with 1% toluidine blue. Semi-thin sections were glued to araldite blocks and detached from the glass slide by freezing-thawing cycles using liquid nitrogen. Ultrathin sections (0.07 μm) were prepared with an Ultracut and then stained with lead citrate. Finally, photomicrographs were obtained under an FEI microscope (Tecnai Spirit G2) using a digital camera (Soft Imaging System, Olympus).

### Nile-Red Staining

Neutral lipid quantification was performed with Nile-Red (Greenspan et al., 1985). Cells (10⁵) were washed twice in PBS and then incubated with 25 μg/ml of Nile-Red (Invitrogen) in PBS (15 min, 4° C.). After incubation, cells were filtered through a 30 μm sieve (CellTrics, Partec). Fluorescent emission (525 nm) was registered in the FL2 channel by a Beckman Coulter Flow Cytometer (Cytomics™ FC 500). Data files were analyzed using the FlowJo flow cytometer analysis software.

### Oil-Red Staining

Lipid droplets were stained by Oil-red (Kutt and Tsaltas, 1959). A stock of Oil-red solution was prepared in 2-propanol (0.3%), vortexed and filtered through a 0.8 μm sieve before staining. The cells (10⁵) grown on the 6-well plates were washed twice in PBS and fixed with 4% of formaldehyde (Panreac) for 30 min. After fixation, cells were washed 3 times in PBS and stained with Oil-red (60 volumes of stock solution: 40 volumes of water). Cells were washed with cold PBS (more than 3 times) and then stained in filtered hematoxylin (Sigma-Aldrich) for 2 min. After staining, cells were washed with PBS and pictures were taken under a light contrast microscope (Leica DMIL) using the Leica Application Suite version 2.4.0 R1 software (Leica Microsystems, Switzerland).

### Reverse Transcriptase PCR (RT-PCR)

Gene expression was performed by RT-PCR techniques. RNA was isolated by using RNeasy® (Qiagen) according to the manufacturer's instructions. RNA quantification was measured by a Nanodrop platform (Thermo Scientific). Then 500 ng of total RNA was converted into cDNA with oligo-dT by using the Advantage® RT-for-PCR kit (Clontech) and following the manufacturer's protocol. Next 5 μl of cDNA were subjected to PCR amplification using Biotag™ DNA polymerase (Bioline) following the manufacturer's instructions. The PCR amplification cycles included [(94° C., 5′); 30 cycles at (94° C., 20′) (56° C., 20″) (72° C., 40″); (72° C., 1′)]. The primers for each gene were designed using Primer3, (http://frodo.wi.mit.edu/primer3/). Subsequently, a blast analysis was performed to test primer specificity using the Primer-Blast tool: (http://www.ncbi.nlm.nih.gov/tools/primer-blast/). The selected primers are indicated in Table. 1. PCR products were resolved on 1.5% agarose gels (Agarose D-1, low EEO-GQT, Pronadisa), stained with 0.1 μg/ml of ethidium bromide (Sigma-Aldrich), and visualized in an UV-transilluminator (BioRad). Band densitometry was calculated by using the Quantity One software.

qRT-PCR

The qPCR analysis was performed using a LightCycler® platform including all the kit components (Roche). qPCR cycles included: [(94° C., 10′); 40 cycles at (95° C., 10″) (65, 6″) (72° C., 10″)]. RNA normalization was performed with glyceraldehydes-3-phosphate dehydrogenase (GAPDH) (Huggett et al., 2005). Fold induction was calculated by the equation:

2^{-ΔΔCt}, ΔΔCt=[(Ct_{gene of interest}−Ct_{internal control}) sample A−(Ct_{gene of interest}−Ct_{internal control}) sample B.

### Confocal Microscopy

Images were acquired with a Leica TCS SP2 AOBS (Leica Microsystems Heidelberg GmbH, Mannheim, Germany) inverted laser scanning confocal microscope using a 63× Plan-Apochromat-Lambda Blue 1.4 N.A. oil objective. The excitation wavelengths for fluorochromes used were 514 nm (Nile-red) and 405 nm (DAPI). The emission apertures for fluorescence detection were (580-680 nm for Nile-red) and (420-470 nm for DAPI). Two-dimensional pseudo color images were obtained with a size of 1024×1024 pixels (step side=0.5 μm). All the confocal images were acquired using the same settings, while the distribution of fluorescence was analyzed using the Leica Confocal “Leica Lite” software, version 2.61.

### Results

### Knock-Out Serum Replacement (KOSR) Triggers Granulogenesis of Human Cancer Cell Lines (HCCLs)

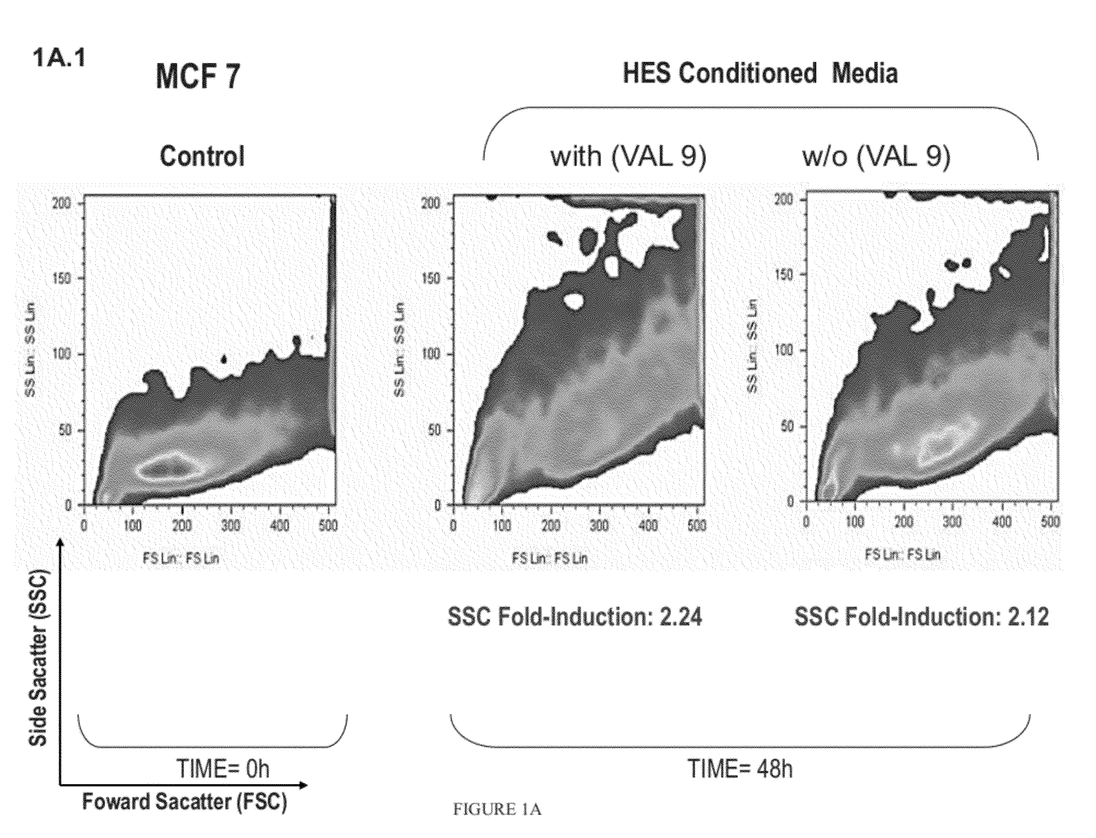

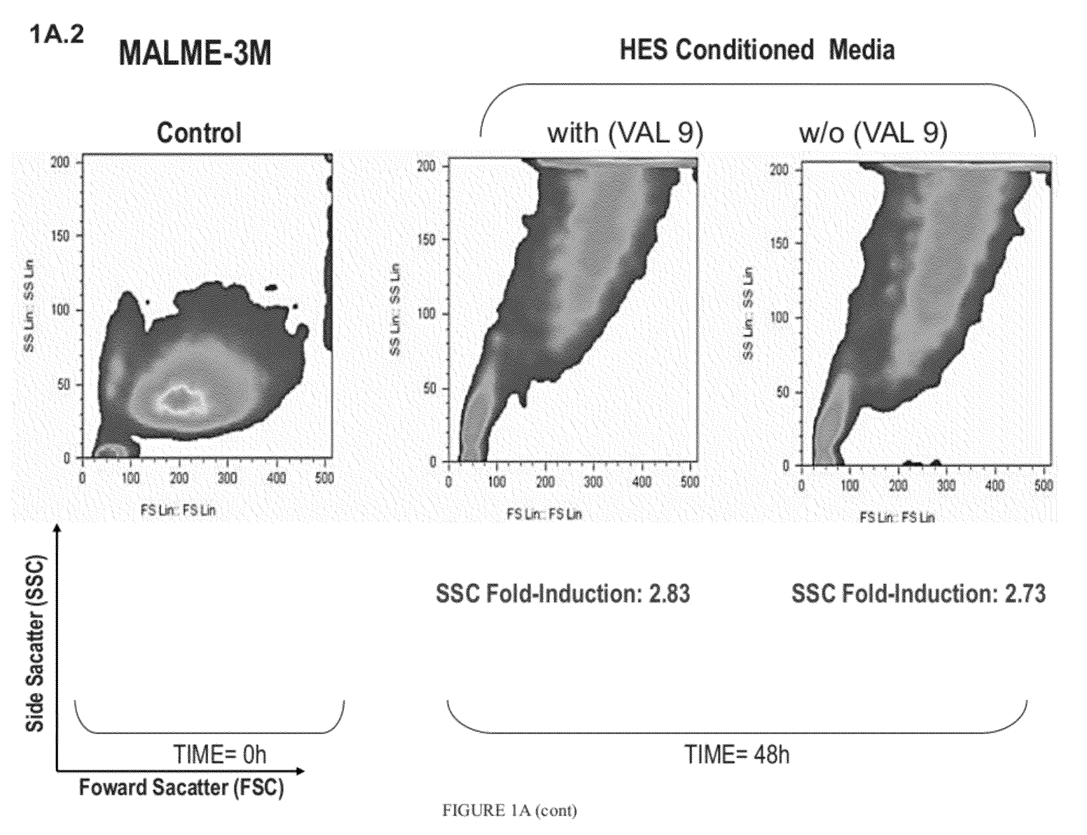

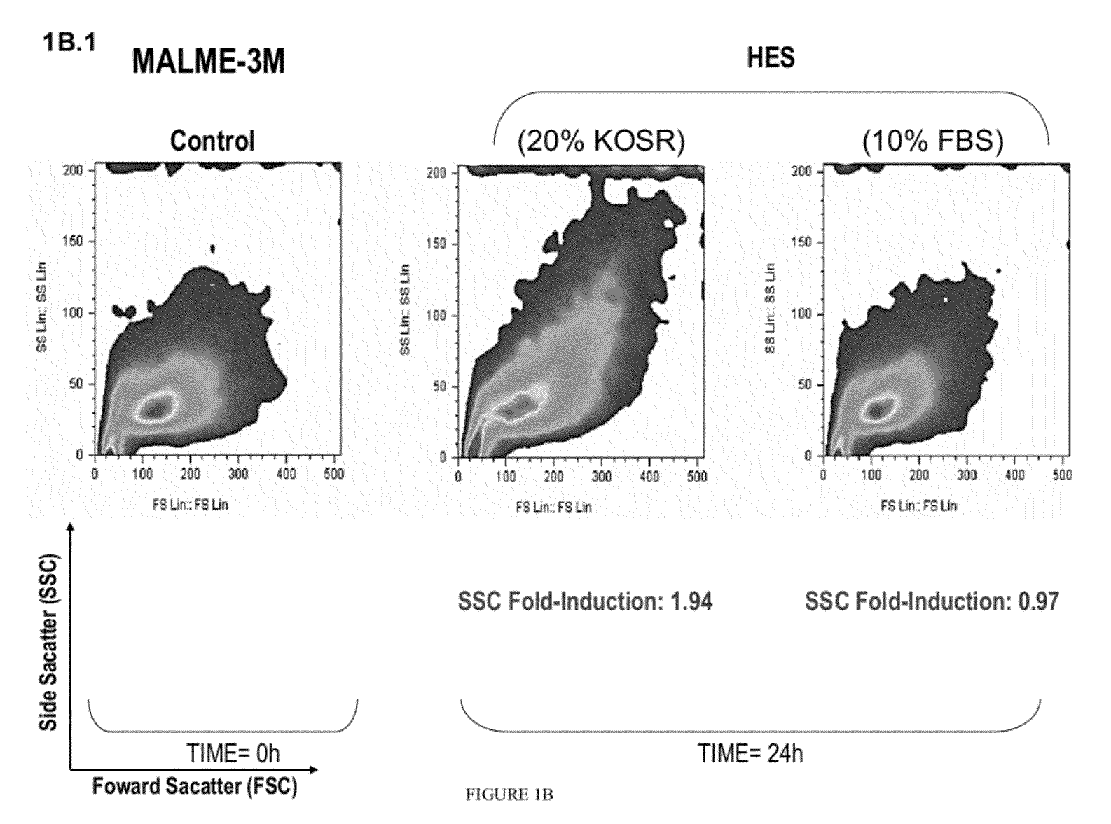

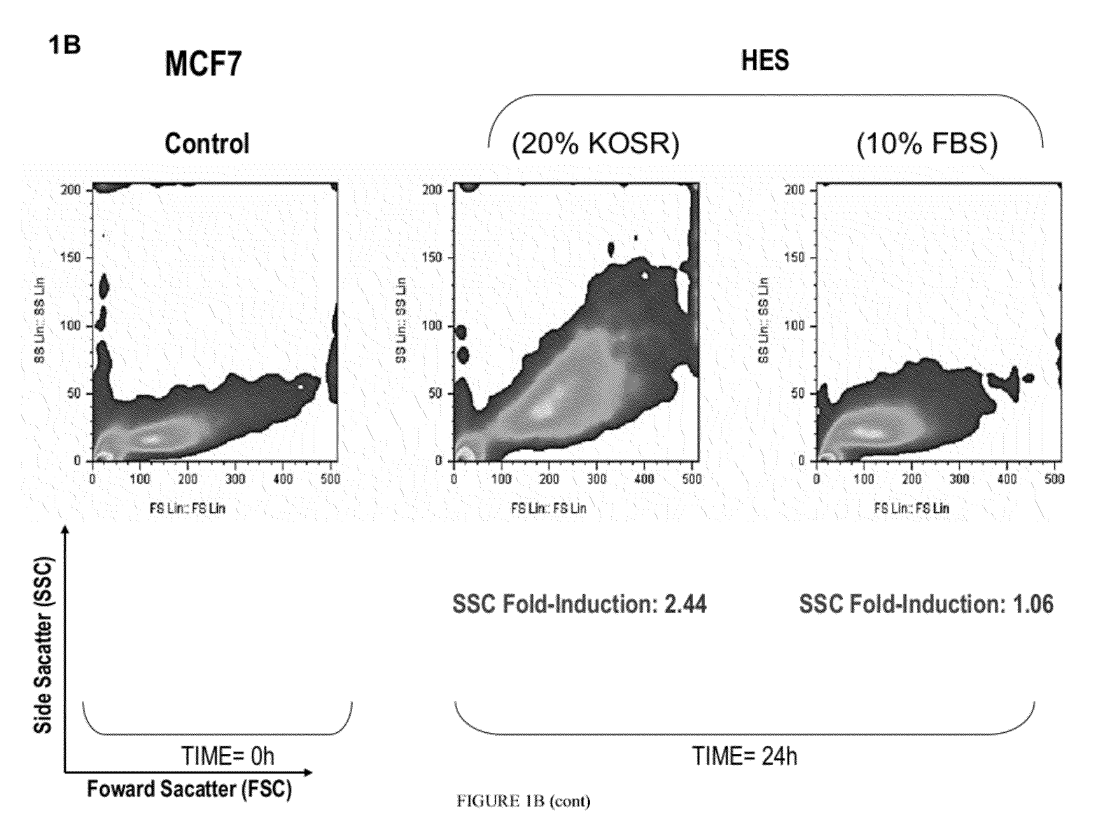

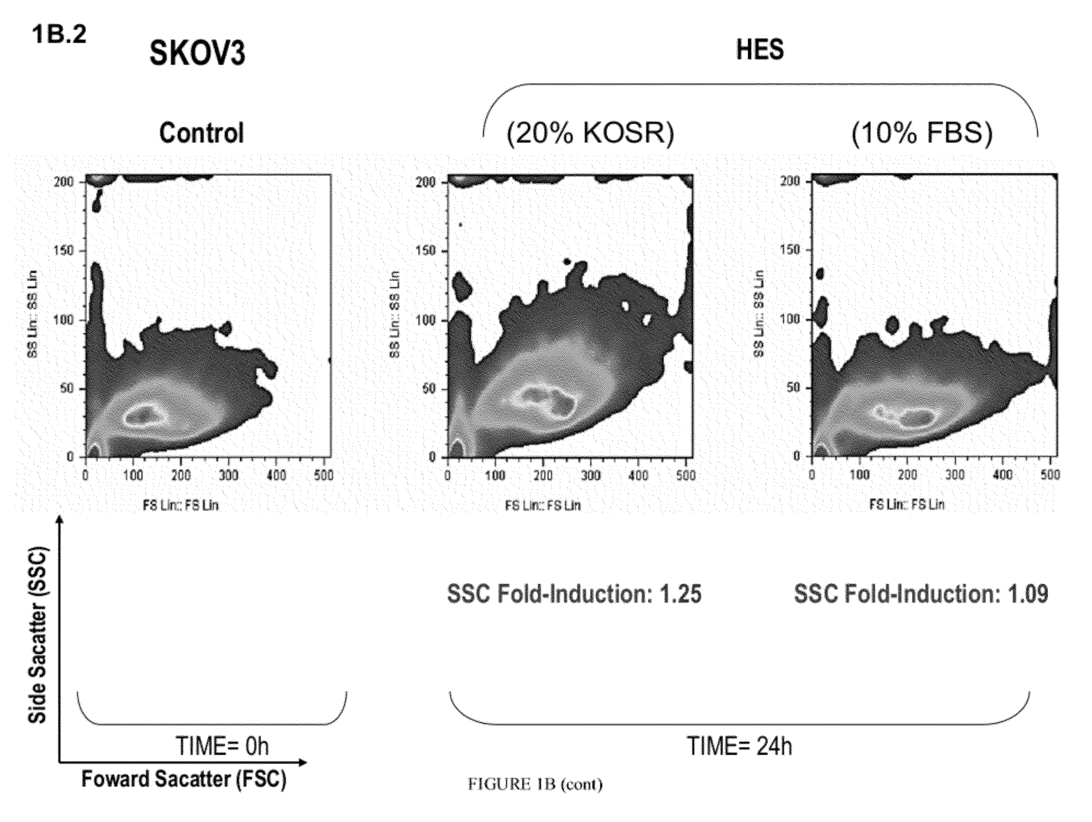

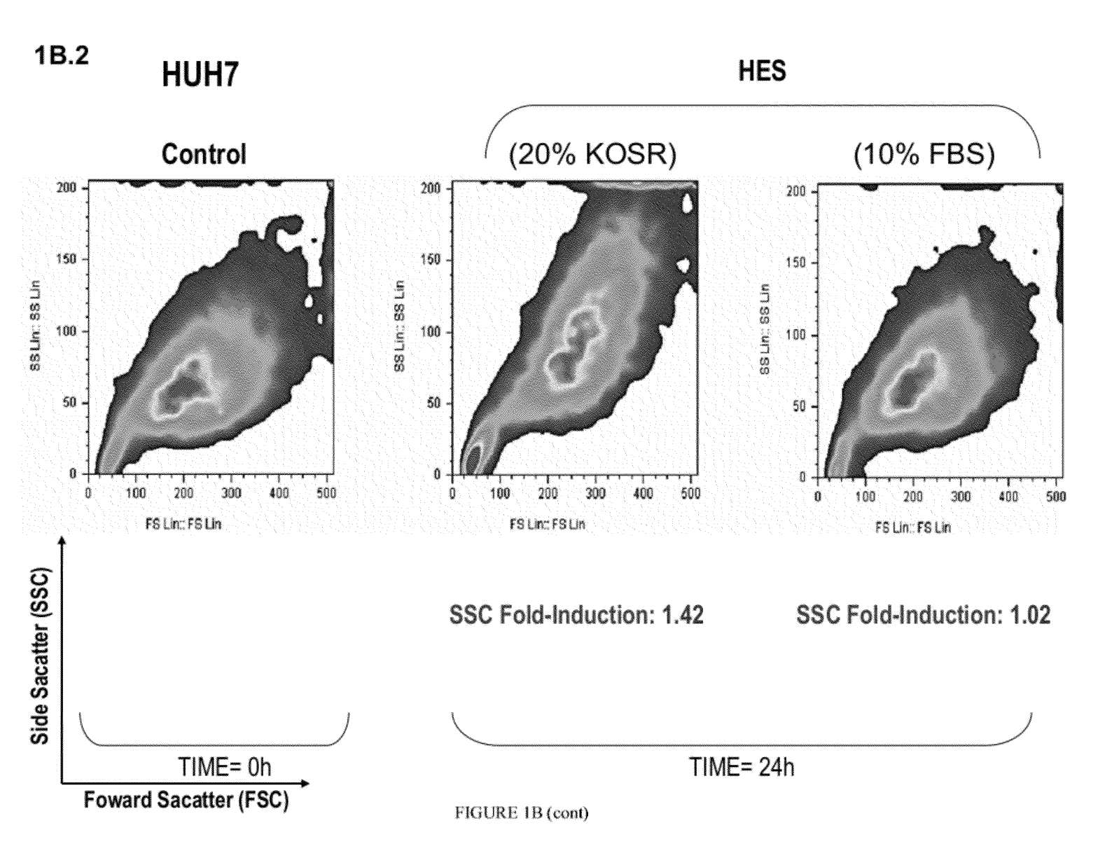

In order to gain insight into the role of human embryonic stem cells (hESCs) conditioned media as a potential source of differentiation factors in human cancer cell lines (HCCLs) (Ruiz-Vela et al., 2009), we examined whether hESC-conditioned media might trigger cellular modifications in HCCLs (MCF-7, MALME-3M, HUH-7 and SK-OV-3) by searching for alterations in the threshold of forward scatter (FSC) and side scatter (SSC) parameters (Terstappen et al., 1991). To accomplish this, we cultured MCF-7 and MALME-3M cells with HES medium containing 20% of KOSR (HES+20% KOSR) conditioned in the presence or absence of hESCs (FIG. 1A). As negative controls, MCF-7 and MALME-3M cells were cultured with RPMI-1640 containing 10% of fetal bovine serum (RPMI+10% FBS). This experiment demonstrated remarkable alterations in the threshold of SSC of MCF-7 and MALME-3M cells (≧2-fold induction at 48 hours) upon incubation with HES+20% KOSR, irrespectively of the presence or absence of hESC (FIG. 1A). Such dramatic changes in the SSC parameter were not observed when MCF-7 and MALME-3M cells were grown in RPMI+10% FBS (FIG. 1A), indicating that the modifications in the SSC parameter are a result of the culture of those cells with HES+20% KOSR.

In order to crossvalidate this finding, we examined the effect of HES+20% KOSR on both HUH-7 and SK-OV-3 cells (FIG. 1B). To assess this, HCCLs were cultured in HES+20% KOSR (FIG. 1B). In addition, KOSR was replaced with 10% FBS (HES+10% FBS) and resulted in no significant alterations, similarly to the situation when HCCLs were cultured in the presence of RPMI+10% FBS (FIG. 1B). It is important to note that HCCLs displayed an increase in the SSC parameter when cells were cultured in the presence of KOSR. These changes in the SSC parameter were not detected when cells were grown in the presence of FBS, indicating that KOSR itself is the factor responsible for triggering alterations in the SSC patterns of HCCLs. Therefore, these results strongly suggest that KOSR might induce granulogenesis in HCCLs (Terstappen et al., 1991).

### KOSR Induces the Biogenesis of Lipid Droplets (LDs) in HCCLs

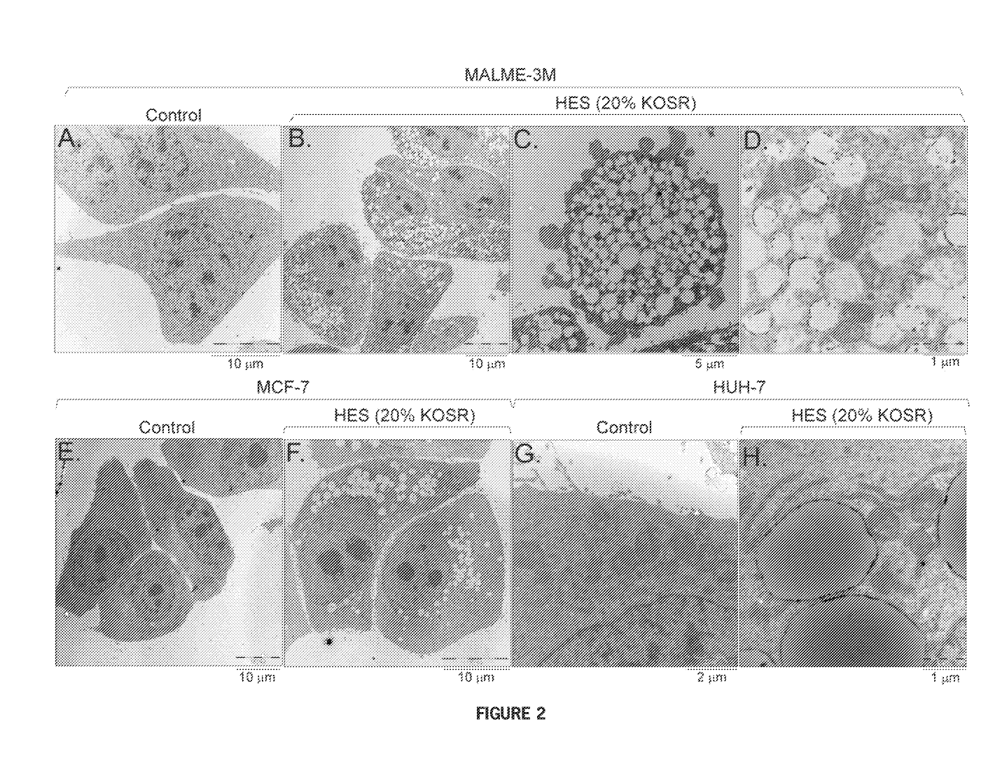

These results encouraged us to wonder about the subcellular modifications induced by KOSR in HCCLs. We performed an electron microscopy (EM) analysis to search for the ultrastructural features responsible for the previously described cellular changes. To this end, MALME-3M and MCF-7 cells were cultured in HES+20% KOSR, whereas control cells were cultured in RPMI+10% FBS. EM studies demonstrate a massive accumulation of spherical structures when cells are cultured with HES+20% KOSR (FIG. 2B-D, F and H), which were not be detected in the control cells cultured in RPMI+10% FBS (FIGS. 2A, E and G). Such spherical structures are considered typical hallmarks of lipid droplets (LD) (Farese and Walther, 2009), indicating that the main observable ultrastructural feature is the biogenesis of LDs. These results are in agreement with the increase noted in the SSC parameter (FIGS. 1A and B), which establishes granulogenesis (Suzuki et al., 1991; Terstappen et al., 1991).

It is important to observe that an LD core is composed of neutral lipids, such as triacylglycerols (TGAs), surrounded by a monolayer of phospholipids (Farese and Walther, 2009). TGAs are produced from the glycerol-phosphate pathway in which fatty acid moieties are added sequentially to a glycerol backbone (Farese and Walther, 2009). In the final step of TGA synthesis, diacylglycerol and fatty acyl CoAs are converted into TGAs by acyl CoA:diacylglycerol acyltransferases (DGAT), which are localized in mitochondria-associated membranes (Farese and Walther, 2009). We detected an accumulation of mitochondria in close proximity to LDs (FIG. 2H), suggesting the biosynthesis of TGAs upon KOSR incubation.

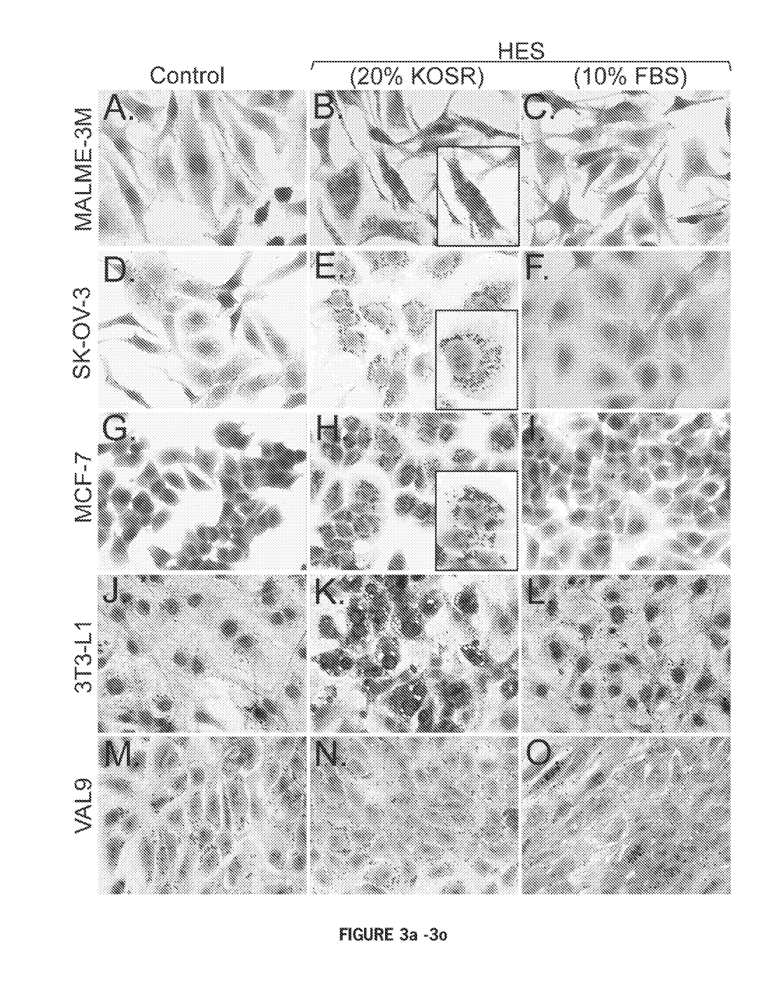

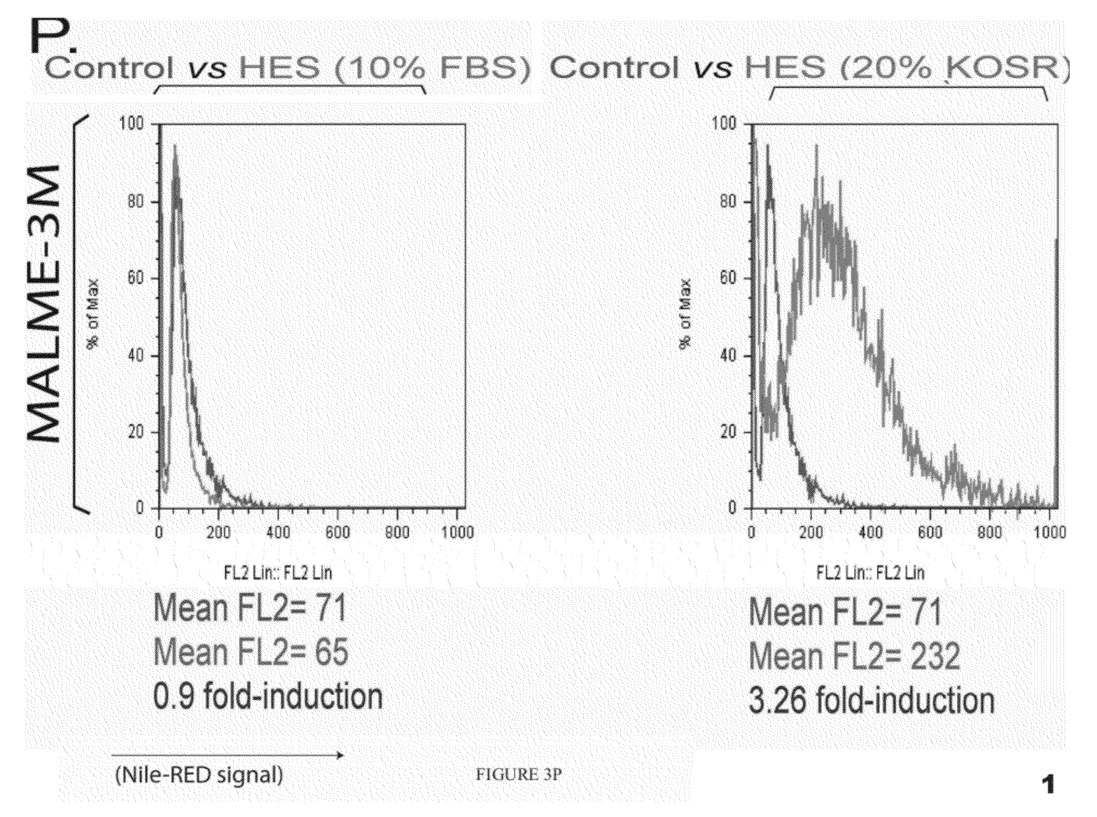

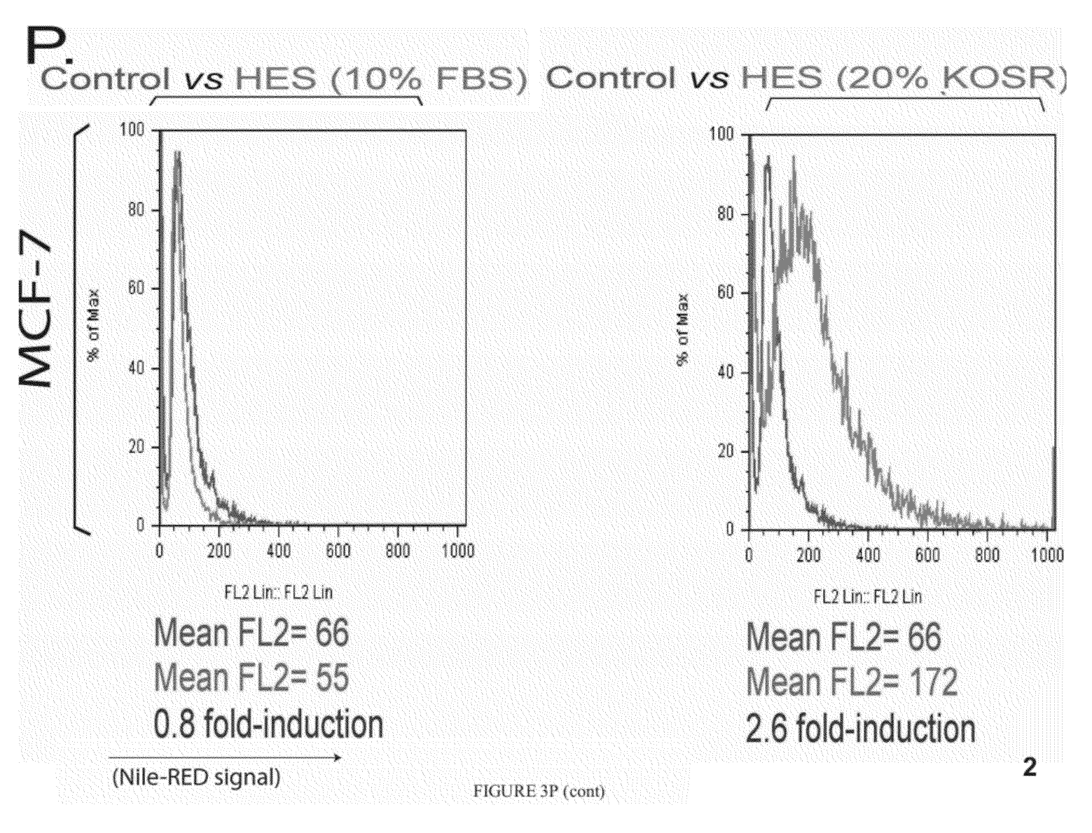

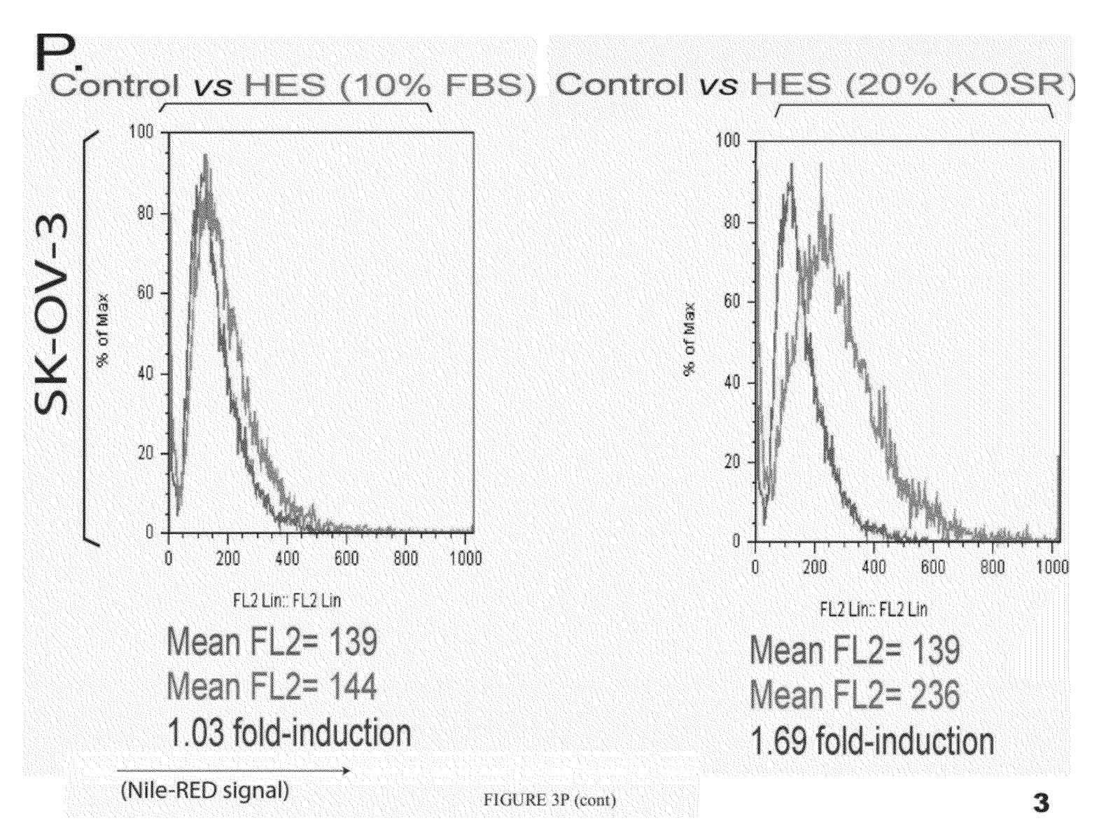

To validate the accumulation of LDs after incubating cells with KOSR, we employed the Oil-RED dye that specifically binds to neutral lipids (Kutt and Tsaltas, 1959). To this end, HCCLs were cultured with HES+20% KOSR or HES+10% FBS for 48 hours, and were stained by Oil-RED and hematoxylin (FIG. 3A-O). As a positive control, we used the preadipocyte cell line (3T3-L1) that underwent accumulation of LDs after incubation with KOSR (FIG. 3J-L). Nonetheless, the human embryonic stem cell (hESC) VAL9 did not show a detectable accumulation of LDs after KOSR incubation, and was used as a negative control (FIG. 3M-O). Furthermore, we quantified the accumulation of LDs in HCCLs in response to HES+20% KOSR incubation. For the purpose of assessing this, we employed Nile-RED, which is a fluorescent dye that binds specifically and is used for LD quantification (Greenspan et al., 1985). HCCLs were cultured with HES+20% KOSR for 48 hours, and were analyzed by Nile-RED staining through flow cytometry analysis. Incubation of HCCLs with HES+20% KOSR led to a marked increase in FL2 intensity if compared to the controls (FIG. 3P). Notably, MALME-3M cells responded very efficiently to LDs accumulation, showing more than a 3-fold induction after incubation with HES+20% KOSR. Accumulation of LDs in cancer cell lines has been previously described (Shiu and Paterson, 1984). However, our results unequivocally demonstrate for the first time that HCCLs accumulate LDs in response to KOSR incubation. On the basis of this, we hypothesized that HCCLs could transdifferentiate toward adipocytes in response to KOSR since a massive accumulation of LDs in the cell is a hallmark of adipocyte differentiation (Mersmann et al., 1975).

Adipogenic Transdifferentiation of HCCLs is Associated with PPARG1 Induction

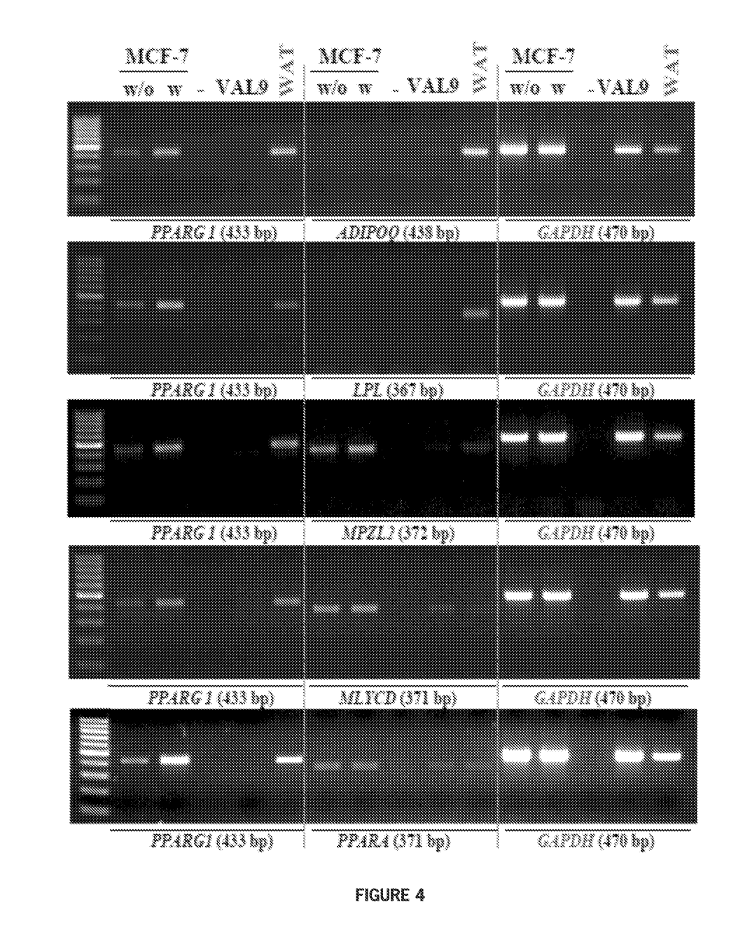

The transcriptional cascade required for adipocyte differentiation centers on the expression and activation of PPARG1, a lipid-activated nuclear hormone receptor that serves as the master transcriptional regulator of adipogenesis (Tontonoz and Spiegelman, 2008). To better understand the molecular mechanism of KOSR-induced LD biogenesis in HCCLs, we examined the expression of several PPARG1-downstream effector genes (such as PPARG1 itself, and ADIPOQ, MPZL2, LPL, PRMD16, ELOVL3, MLYCD and PPARA) (Seale et al., 2007). For this purpose, we cultured MCF-7 cells with HES+20% KOSR with (w) or without (w/o) KOSR, and examined the expression of the adipogenic genes by RT-PCR techniques (FIG. 4). Notably, an up-regulation of PPARG1 was detected after KOSR incubation with no apparent effect on the expressions of ADIPOQ, LPL, MPZL2, PPARA and MLYCD (FIG. 4). Furthermore, we performed a quantitative qRT-PCR analysis of the PPARG1 expression which exhibited a mean 3.34-fold induction (SD=0.47) in PPARG1 if compared to the cells cultured w/o KOSR. It is significant to note that the PPARG1 up-regulation was crossvalidated in the MALME-3M cells in time-course experiments. These experiments indicate that the PPARG1 expression is induced with 20% KOSR. In general terms, this data set indicates that PPARG1 is up-regulated in HCCLs after KOSR incubation. As PPARG1 is responsible for the transdifferentiation of committed cells into adipocytes (Hu et al., 1995), our data suggest that KOSR could trigger the adipogenic transdifferentiation of HCCLs.

Transdifferentiation of HCCLs into Adipocyte-Like Cells Induced by the Fatty Acid-Rich Albumin Fraction (albuMAX)

Our results prompted us to consider the possible transdifferentiating agents in the KOSR formulation responsible for triggering the adipogenic transdifferentiation in HCCLs. We dissected out the KOSR formulation and found that the fatty acid-rich albumin fraction (albuMAX) represents the major component of KOSR (Garcia-Gonzalo and Izpisua Belmonte, 2008). It is important to note that the genetic defect of the human albumin gene (ALB) causes lipodystrophy, characterized by a lack of adipose tissue (Kallee, 1996). Consequently, our primary candidate to be tested as an adipogenic inductor was albuMAX (Garcia-Gonzalo and Izpisua Belmonte, 2008).

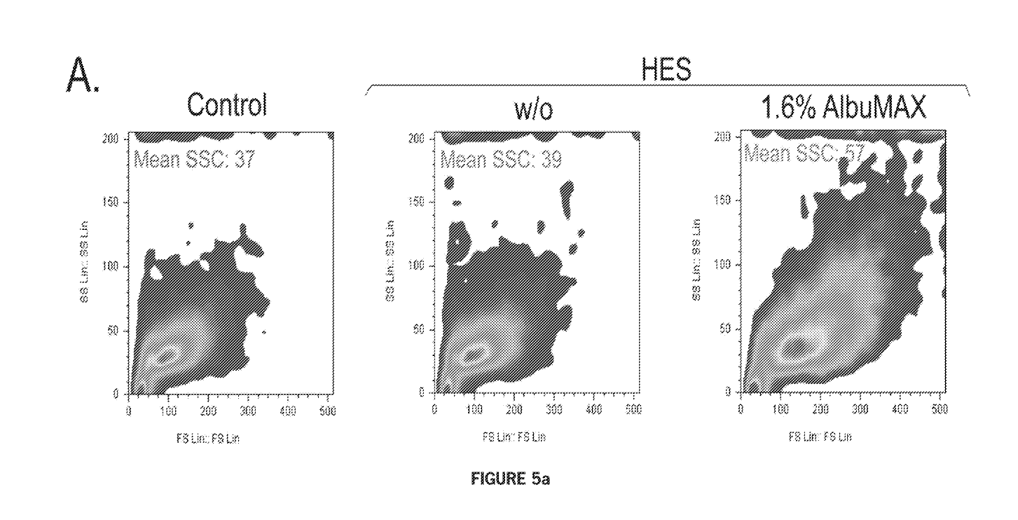

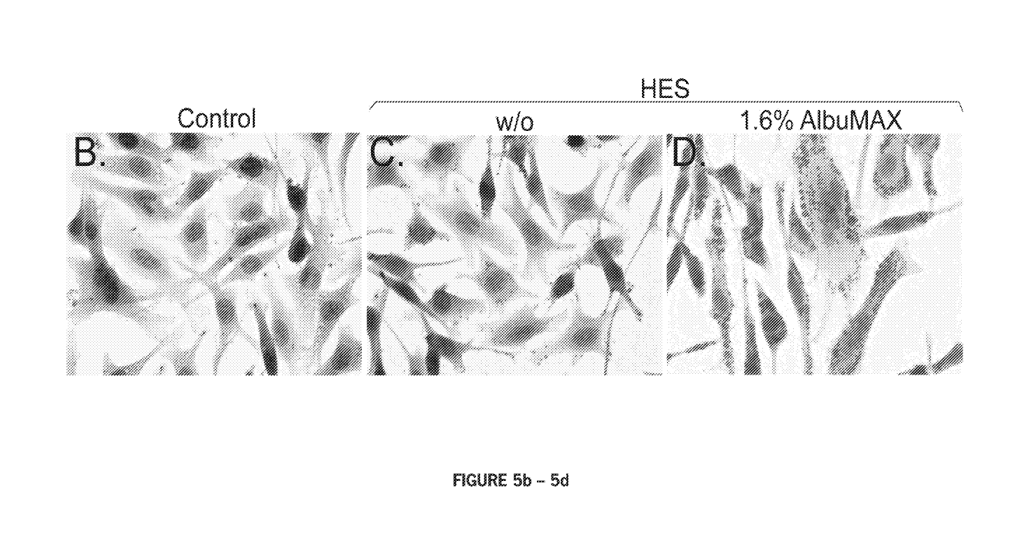

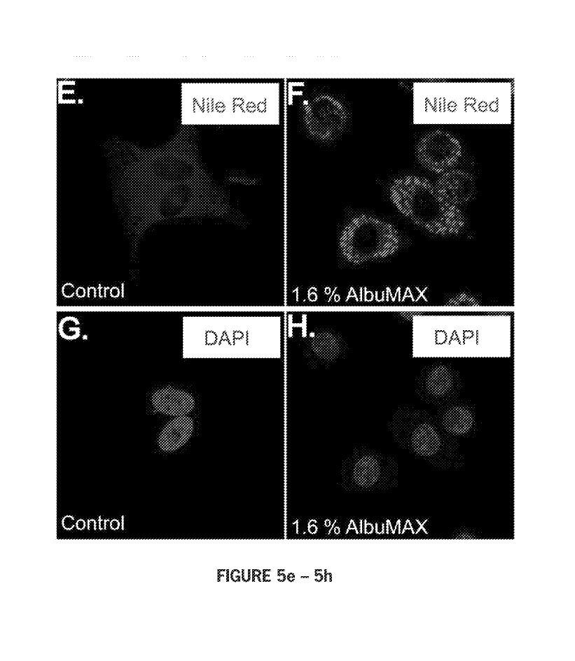

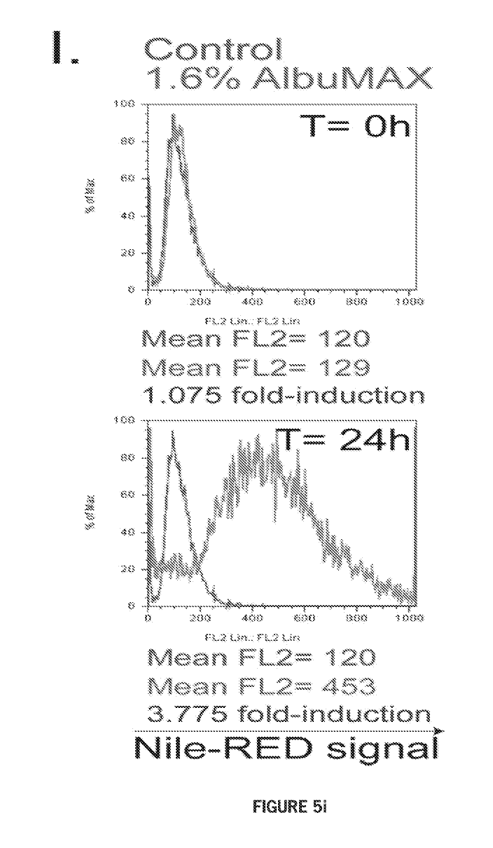

To examine the effect of albuMAX on HCCLs, we used MALME-3M cells since this cell type responds very efficiently to both KOSR-induced LD accumulation (FIGS. 2 and 3) and KOSR-induced PPARG1 up-regulation. We cultured MALME-3M cells in the presence (w) or absence (w/o) of 1.6% of albuMAX, this being the amount of this component in the 20% KOSR medium (Garcia-Gonzalo and Izpisua Belmonte, 2008). As a negative control, we also cultured cells in the presence of RPMI+10% FBS. After a 48-hour incubation, the alterations in the SCC profile were assessed (FIG. 5A), and an analysis of LD accumulation was performed using Oil-RED (FIG. 5B-D) and Nile-RED staining (FIG. 5E-H). AlbuMAX-induced transdifferentiation was also quantified by flow cytometry and showed an accumulation of LDs in MALME-3M cells (FIG. 5I). Furthermore, PPARG1 up-regulation was also up-regulated in a time-course analysis after incubating MALME-3M cells with albuMAX, thus confirming that albuMAX incubation enforces the adipogenic transdifferentiation of MALME-3M cells. All these read-outs were in MCF-7, HUH-7 and SK-OV-3 (data not shown). In general, these results indicate that albuMAX works as an efficient adipogenic transdifferentiating agent in HCCLs.

### Linoleic, Oleic, Palmitoleic and Petroselinic Unsaturated Fatty Acids are Responsible for the Adipogenic Transdifferentiation in HCCLs

AlbuMAX is considered a fatty acid-albumin complex (Garcia-Gonzalo and Izpisua Belmonte, 2008) formed by unsaturated fatty acids (Davis and Dubos, 1947). These data, together with the role of nitro-linoleic and hydroxy-oleic acids as PPARG ligands (Schopfer et al., 2005; Yokoi et al., 2010), led us to hypothesize that unsaturated fatty acids are candidate transdifferentiating molecules.

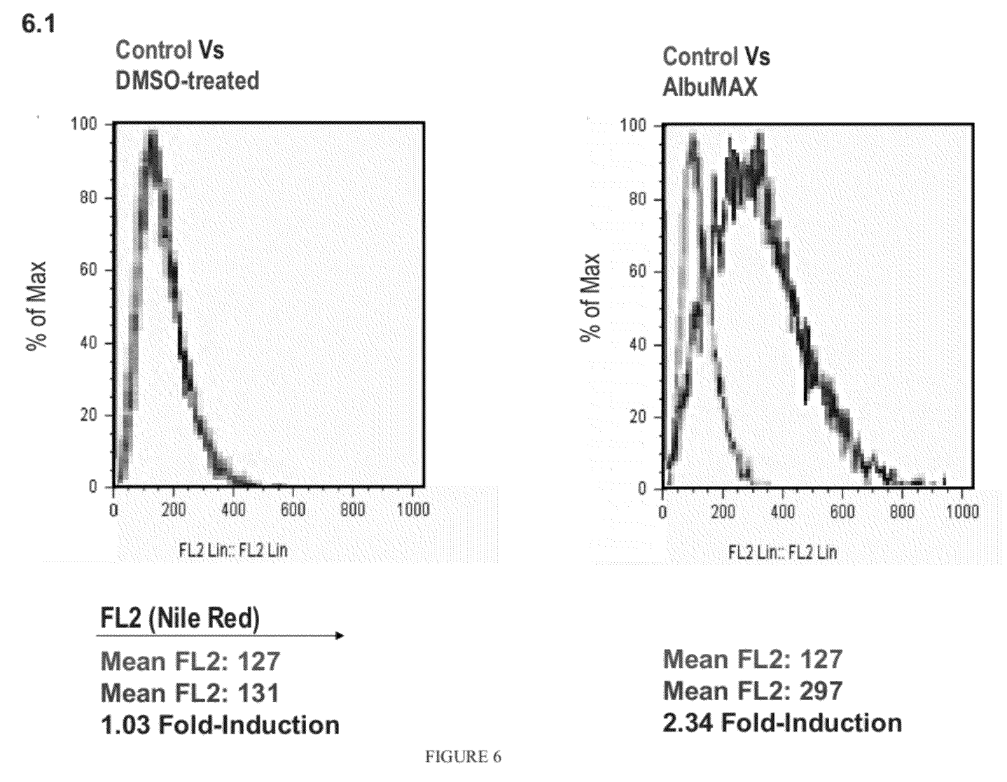

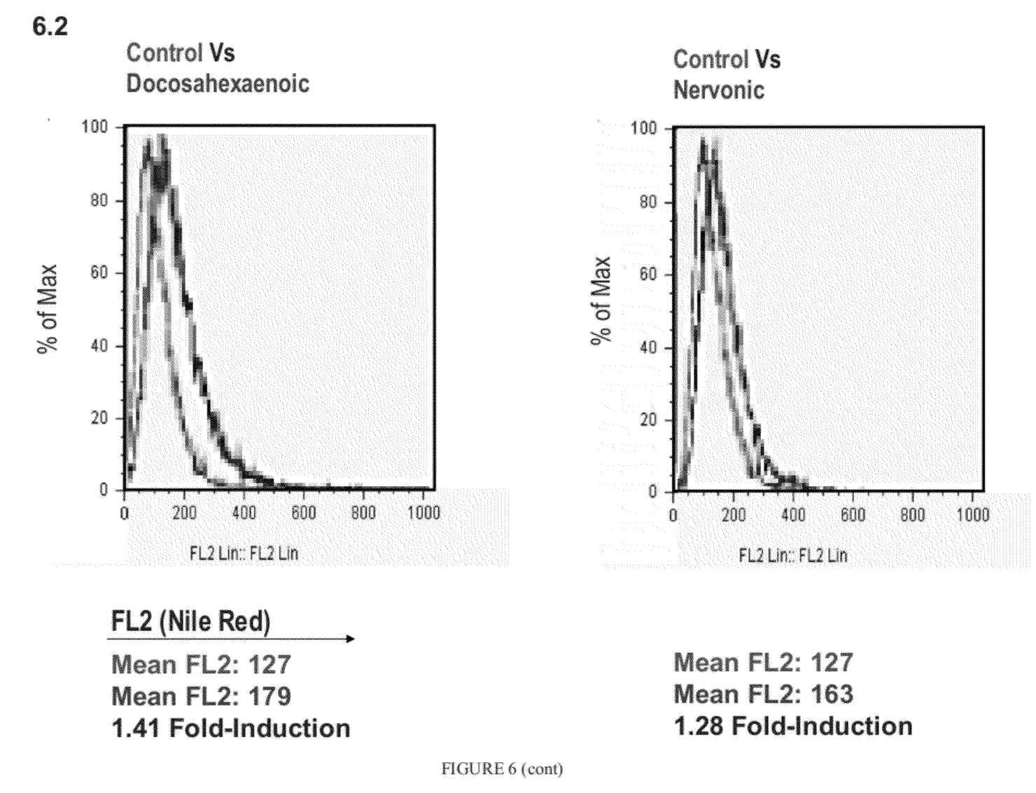

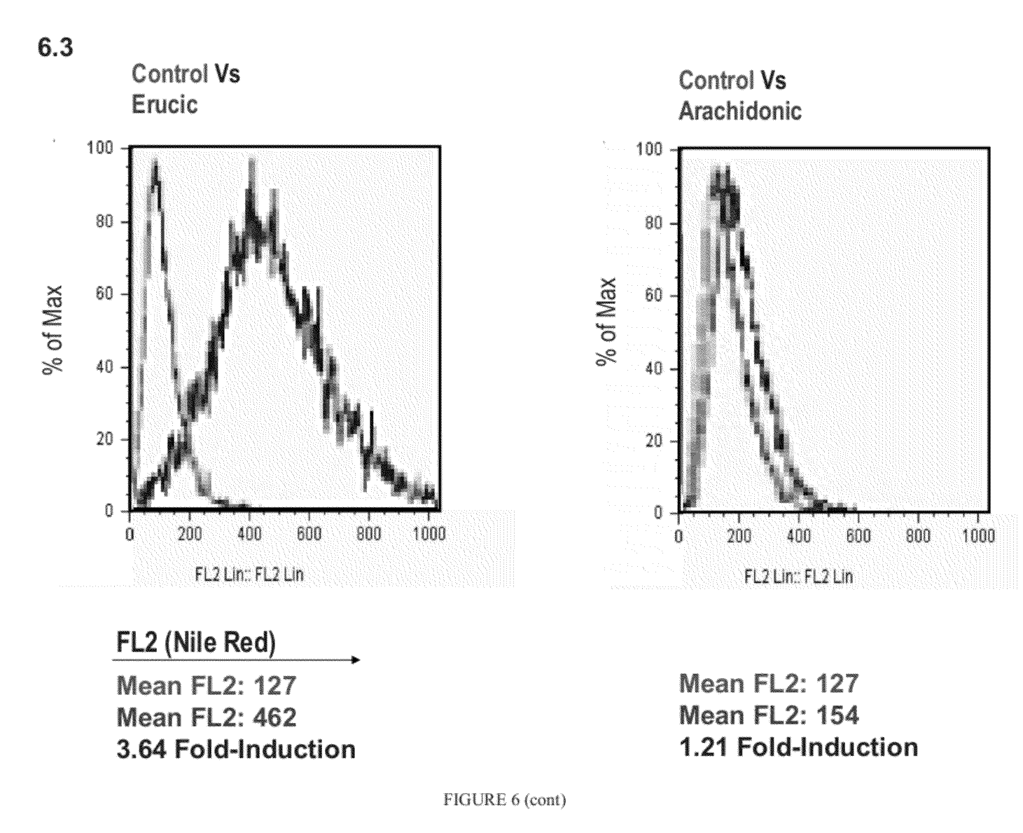

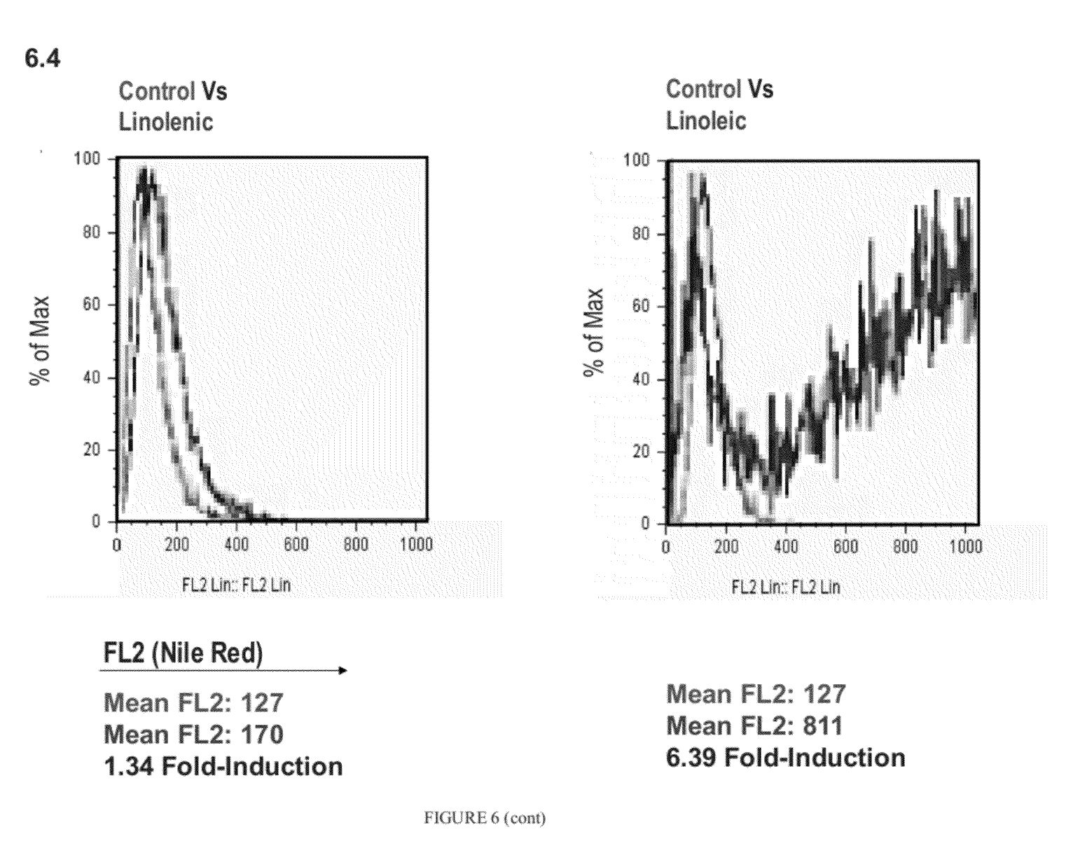

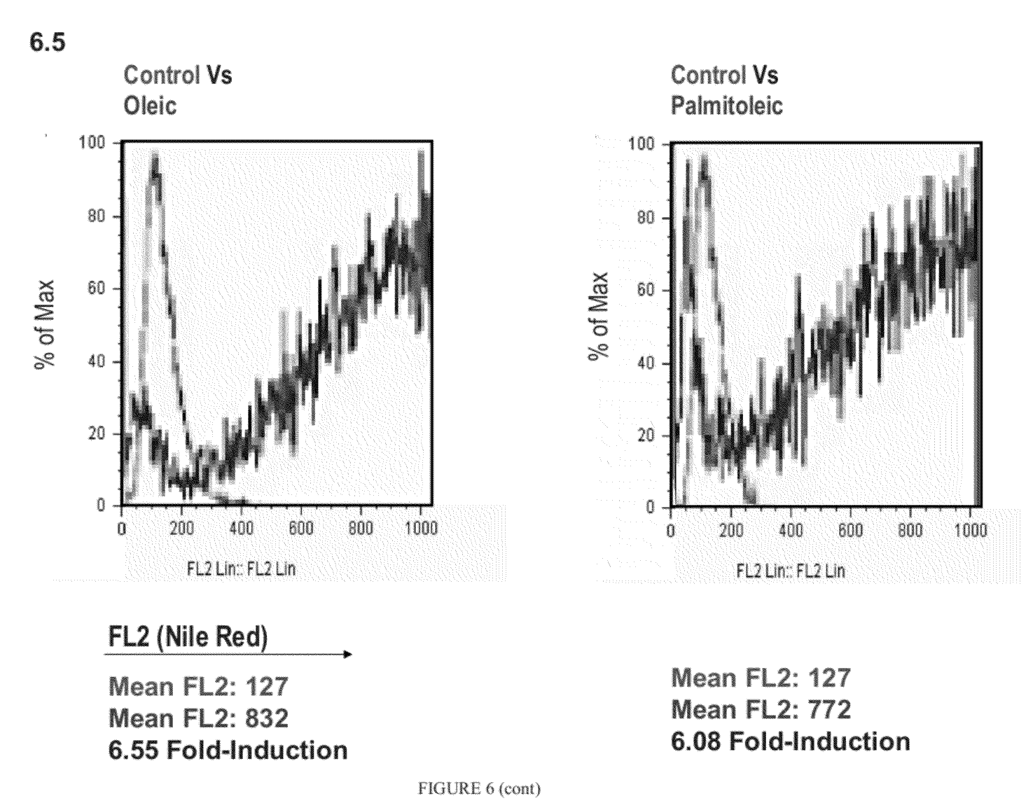

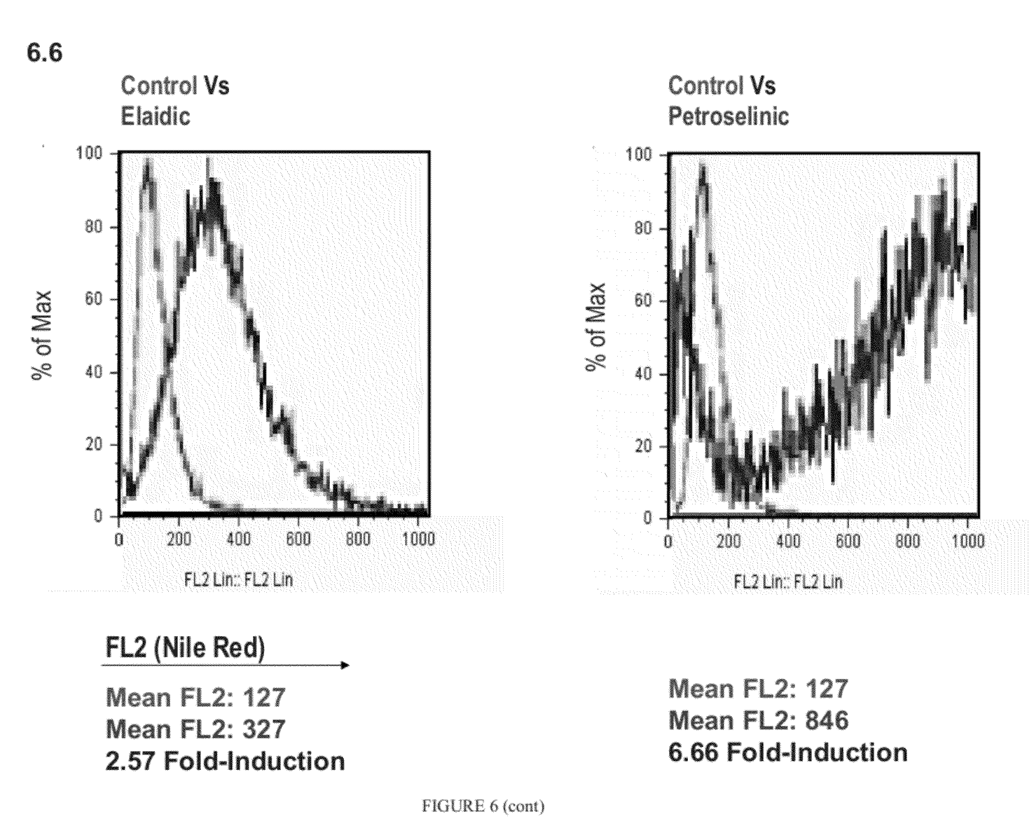

To confirm this, we initially screened 10 mono- and polyunsaturated fatty acids and their ability to induce LDs in MALME-3M cells. Accumulation of LDs was quantified by Nile-RED staining after incubating cells with mono- and polyunsaturated fatty acids. As a negative control, we incubated cells in the presence of RPMI+10% FBS w/o unsaturated fatty acids and with the vehicle DMSO. Cells were also grown in the presence of albuMAX, and were used as a positive control. After a 12-hour incubation period, LD accumulation was induced by palmitoleic, oleic, petroselinic, elaidic, erucic, and linoleic acids (FIG. 6), but not by docosahexaenoic, nervoic, arachidonic and linolenic acids. One important finding was that all the monounsaturated fatty acids tested in MALME-3M cells induced LDs, whereas the polyunsaturated fatty acids induced only a marginal accumulation of LDs, except for linoleic acid (FIG. 6). Furthermore, only linoleic (L), oleic (O), palmitoleic (P) and petroselinic acids (T) very efficiently induced the generation of LDs (a fold induction of ≧6) (FIG. 6). Oleic acid has been previously used to induce lipid droplets in macrophages (Chen et al., 2002) and monocytes (Suzuki et al., 1991). However, our experiments show for the first time that specific unsaturated fatty acids (L, O, P and T) trigger efficient LD accumulation in melanoma cells.

In order to search for an even more efficient transdifferentiating cocktail of unsaturated fatty acids, we combined L, O, P and T, and tested them in MALME-3M and HUH-7 cells. The results obtained indicate that the combination of P, O and L (POL) in MALME-3M; and P, O and T (POT) in HUH-7 prove to be the most efficient cocktails as they induce a ≧5- and ≧2.75-fold induction, respectively, after an 8-hour incubation period. These cocktails (POT and POL) were also tested in breast adenocarcinoma MCF-7 and preadipocyte 3T3-L1, and induced a ≧5- and ≧4-fold induction, respectively (data not shown). Thus, these experiments indicate that POT is the most efficient adipogenic transdifferentiation cocktail in both HUH-7 and MCF-7 cells, whereas POL appears to be most efficient in MALME-3M cells and preadipocyte 3T3-L1. These differences in adipogenic responses may reflect the differences in the downstream effector pathways triggered by the distinct unsaturated fatty acids. It is interesting to note that linoleic acid, but not linolenic acid, triggers LD accumulation in MALME-3M and HUH-7 (FIG. 6). These findings are important since linoleic acid structurally resembles linolenic acid, but lacks only one unsaturation at the position ω3 (http://www.lipidmaps.org/), indicating the specificity of the transdifferentiation process induced by linoleic acid.

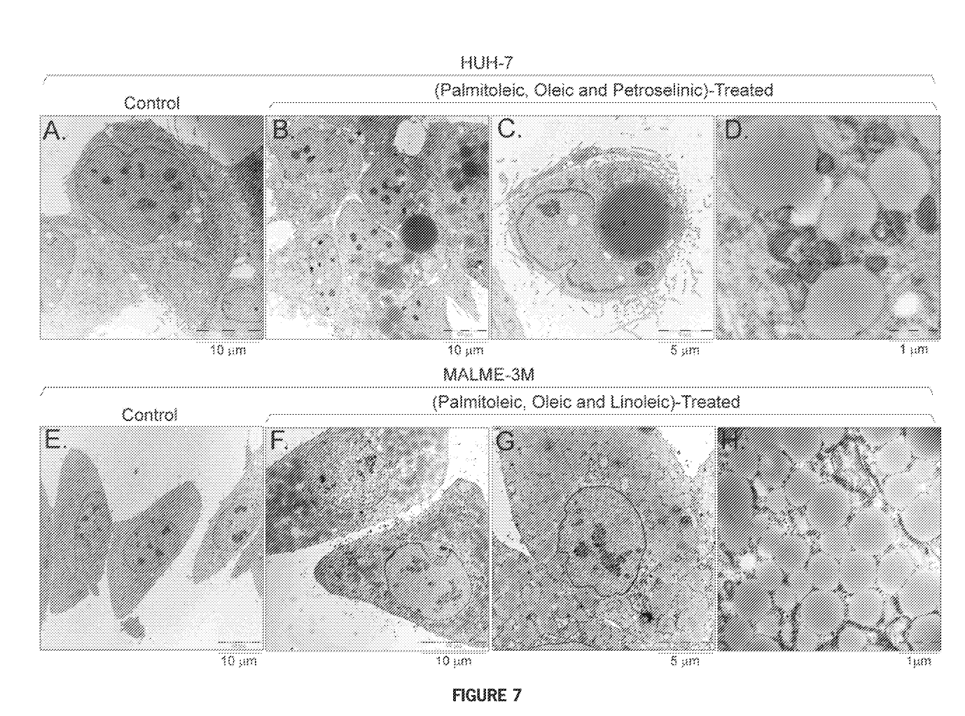

Since P, O and L have been shown to associate directly with albumin (Davis and Dubos, 1947; Lafond et al., 1994; Thomas et al., 1995), we assayed whether the POL cocktail synergizes with human recombinant albumin to induce LDs. MALME-3M cells were incubated in the presence of POL, or with recombinant albumin only, and in combination with both factors (POL: albumin). After a 12-hour incubation period, the neutral lipids contained in the LDs were quantified by Nile-RED. The POL cocktail induced a similar fold induction to that generated by the combination of POL plus recombinant albumin, while albumin itself presented no adipogenic properties (data not shown). EM studies further demonstrate a massive multillocular and an eventual unilocular LDs formation in close proximity to mitochondria and the endoplasmic reticulum in HUH-7 and MALME-3M upon incubation with unsaturated fatty acid cocktails (FIG. 7A-H). These data confirm that specific fatty acids, but not albumin itself, are responsible for HCCLs transdifferentiation into adipocyte-like cells.

### Discussion

Despite the important accomplishment of differentiation therapy in the identification of novel genes/molecules to trigger terminal transdifferentiation of human cancer cells, many key questions remain unanswered. In this study, lipid-albumin complexes (albuMAX) were found to induce the transdifferentiation of HCLLs toward adipocytes, and also led to a provider of transcriptional signaling linked to PPARG1 up-regulation. These findings indicate that the connection between albuMAX and pluripotency (Garcia-Gonzalo and Izpisua Belmonte, 2008) is surprisingly more complex than previously anticipated. In addition to the albuMAX function as a factor required for maintaining self-renewal and pluripotency in hESC (Garcia-Gonzalo and Izpisua Belmonte, 2008), a role for albumin (Kallee, 1996) and the associated lipids (Davis and Dubos, 1947; Lafond et al., 1994; Thomas et al., 1995) have been seen to participate in adipogenesis in other cell types (Schopfer et al., 2005). At this point, it is not clear whether albuMAX-induced pluripotency is due to a direct target or if it is possibly a specific effect on hESCs. How all the targets of albuMAX are sensed in hESC versus HCCLs is part of this puzzle.

AlbuMAX very efficiently triggers the biogenesis of the LDs in HCCLs, as detected by several techniques including electron microscopy and oil-RED staining, and were quantified by flow cytometry using the nile-RED dye. AlbuMAX-induced transdifferentiation was also accompanied by PPARG1 up-regulation. We demonstrate that albuMAX-induced PPARG1 up-regulation is very specific since the other genes associated with adipogenesis (i.e., ADIPOQ, MPZL2, LPL, PRMD16, ELOVL3, MLYCD and PPARA) were not up-regulated. These findings are important since most of the above-mentioned genes have been discovered in murine cells, and our results indicate important differences between adipogenesis in mice versus humans.

Encouraged by the results obtained with albuMAX in HCCLs, we decided to determine the specific molecules responsible for the adipogenic transdifferentiation process. Given that several fatty acids form a complex with albumin (Davis and Dubos, 1947; Garcia-Gonzalo and Izpisua Belmonte, 2008; Lafond et al., 1994; Thomas et al., 1995), we screened the unsaturated fatty acid family and its ability to induce adipogenic transdifferentiation. With this approach, we discovered that linoleic, oleic and palmitoleic acids, but not albumin, induce LD accumulation very efficiently, which is in agreement with the role of linoleic, oleic and palmitoleic as PPARG ligands (Kliewer et al., 1994; Michaud and Renier, 2001; Sauma et al., 2006). Thus, our results indicate that certain HCCLs exhibit a surprisingly high degree of plasticity in response to linoleic, oleic and palmitoleic acids, which suggests adipocyte transdifferentiation. In addition, our results indicate that the use of poly- and monounsaturated fatty acids cocktails improves their efficiency, and suggest the possibility of inducing terminal differentiation more efficiently and blocking cancer progression.

Since the transdifferentiating lipids in cancer (e.g., all trans-retinoic acids (Waxman, 2000) or the linoleic acid isomer 9Z-11E (Kuniyasu et al., 2006)) mediate their functions together with nuclear receptors, pathologic processes such as human cancer should be treated by considering a complex network of interconnected nuclear receptors. Thus, we may consider a “system biology approach” in which enforced transdifferentiation of human cancer cells should be activated by a given combination of transdifferentiating lipids. The elucidation of these novel connections, plus the roles played in adipogenic transdifferentiation, should provide a solid foundation to open up new cell biology avenues for cancer therapy.

### EXAMPLE 2

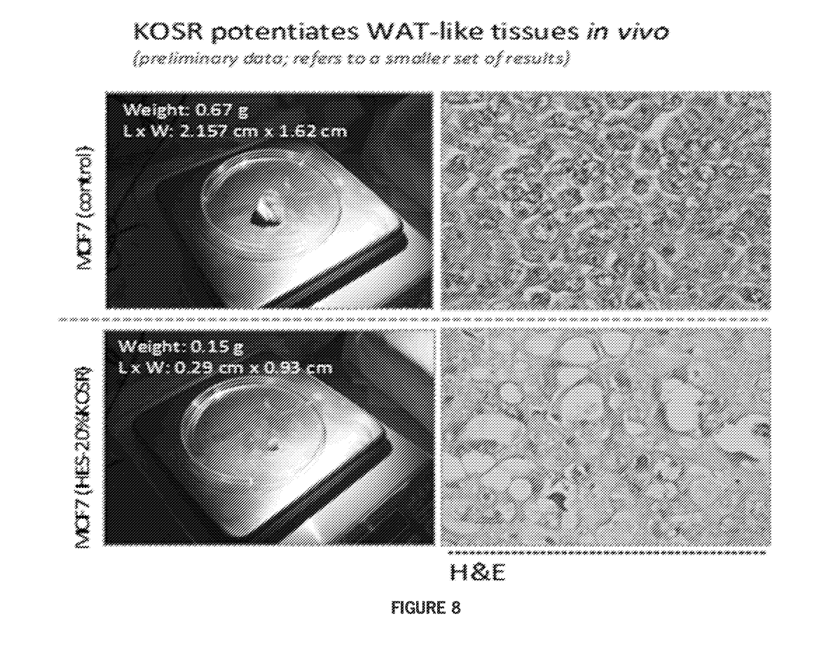

We have conducted an in vivo xenograft model, and shown that AlbuMAX-induced metaplastic adipogenesis of human cancer cells was associated with tumour reduction (FIG. 8), pointing out the possibility to revert the original identity of human cancer cells through transdifferentiation towards a terminal adipogenic phenotype. MCF-7 cancer cells were transdifferentiated towards adipocyte-like cells through incubation with HES media during 14 days, these cells were termed Adipo-MCF-7 cells. In parallel, we grew MCF-7 cancer cells in the presence of RPMI-(10% FBS) used as negative control. Next, we subcutaneously transplanted 10⁵ human cancer cells from each cell type into male NOD/SCID mice for in vivo analysis of tumor progression. After 2 months of transplantation, analysis of tumour cell size in transplanted NOD/SCID mice indicated a profound reduction in tumour growth induced by Adipo-MCF-7 cells compared with their counterpart MCF-7 cells. Notably, H&E analysis in tumours originated from Adipo-MCF-7 cells exhibited features of adipocyte terminal transdifferentiation as evaluated by the presence of condensed nucleus excluded from the cytoplasm and the presence of large lipid droplets resembling to white adipocyte tissues (WAT). Thus, these results indicate that Adipo-MCF-7 cells exhibit an increase potential in adipocyte-like features in vivo, such as the acquisition of hallmarks of terminal differentiation, which are responsible of less tumorigenicity.

### REFERENCES

Chen, J. S., Greenberg, A. S., and Wang, S. M. (2002). Oleic acid-induced PKC isozyme translocation in RAW 264.7 macrophages. J Cell Biochem 86, 784-791.

Davis, B. D., and Dubos, R. J. (1947). The Binding of Fatty Acids by Serum Albumin, a Protective Growth Factor in Bacteriological Media. J Exp Med 86, 215-228.

Farese, R. V., Jr., and Walther, T. C. (2009). Lipid droplets finally get a little R-E-S-P-E-C-T. Cell 139, 855-860.

Forsberg, M., Carlen, M., Meletis, K., Yeung, M. S., Barnabe-Heider, F., Persson, M. A., Aarum, J., and Frisen, J. (2010). Efficient reprogramming of adult neural stem cells to monocytes by ectopic expression of a single gene. Proc Natl Acad Sci USA.

Garcia-Gonzalo, F. R., and Izpisua Belmonte, J. C. (2008). Albumin-associated lipids regulate human embryonic stem cell self-renewal. PLoS One 3, e1384.

Graf, T., and Enver, T. (2009). Forcing cells to change lineages. Nature 462, 587-594.

Greenspan, P., Mayer, E. P., and Fowler, S. D. (1985). Nile red: a selective fluorescent stain for intracellular lipid droplets. J Cell Biol 100, 965-973.

Hu, E., Tontonoz, P., and Spiegelman, B. M. (1995). Transdifferentiation of myoblasts by the adipogenic transcription factors PPAR gamma and C/EBP alpha. Proc Natl Acad Sci USA 92, 9856-9860.

Huggett, J., Dheda, K., Bustin, S., and Zumla, A. (2005). Real-time RT-PCR normalisation; strategies and considerations. Genes Immun 6, 279-284.

Ieda, M., Fu, J. D., Delgado-Olguin, P., Vedantham, V., Hayashi, Y., Bruneau, B. G., and Srivastava, D. (2010). Direct Reprogramming of Fibroblasts into Functional Cardiomyocytes by Defined Factors. Cell 142, 375-386.

Kakizuka, A., Miller, W. H., Jr., Umesono, K., Warrell, R. P., Jr., Frankel, S. R., Murty, V. V., Dmitrovsky, E., and Evans, R. M. (1991). Chromosomal translocation t(15; 17) in human acute promyelocytic leukemia fuses RAR alpha with a novel putative transcription factor, PML. Cell 66, 663-674.

Kallee, E. (1996). Bennhold's analbuminemia: a follow-up study of the first two cases (1953-1992). J Lab Clin Med 127, 470-480.

Kliewer, S. A., Forman, B. M., Blumberg, B., Ong, E. S., Borgmeyer, U., Mangelsdorf, D. J., Umesono, K., and Evans, R. M. (1994). Differential expression and activation of a family of murine peroxisome proliferator-activated receptors. Proc Natl Acad Sci USA 91, 7355-7359.

Kuniyasu, H., Yoshida, K., Sasaki, T., Sasahira, T., Fujii, K., and Ohmori, H. (2006). Conjugated linoleic acid inhibits peritoneal metastasis in human gastrointestinal cancer cells. Int J Cancer 118, 571-576.

Kutt, H., and Tsaltas, T. T. (1959). Staining properties of oil red O and a method of partial purification of the commercial product. Clin Chem 5, 149-160.

Lafond, J., Simoneau, L., Savard, R., and Gagnon, M. C. (1994). Linoleic acid transport by human placental syncytiotrophoblast membranes. Eur J Biochem 226, 707-713.

Mersmann, H. J., Goodman, J. R., and Brown, L. J. (1975). Development of swine adipose tissue: morphology and chemical composition. J Lipid Res 16, 269-279.

Michaud, S. E., and Renier, G. (2001). Direct regulatory effect of fatty acids on macrophage lipoprotein lipase: potential role of PPARs. Diabetes 50, 660-666.

Ruiz-Vela, A., Aguilar-Gallardo, C., and Simon, C. (2009). Building a Framework for Embryonic Microenvironments and Cancer Stem Cells. Stem Cell Rev Rep.

Sauma, L., Stenkula, K. G., Kjolhede, P., Stralfors, P., Soderstrom, M., and Nystrom, F. H. (2006). PPAR-gamma response element activity in intact primary human adipocytes: effects of fatty acids. Nutrition 22, 60-68.

Schopfer, F. J., Lin, Y., Baker, P. R., Cui, T., Garcia-Barrio, M., Zhang, J., Chen, K., Chen, Y. E., and Freeman, B. A. (2005). Nitrolinoleic acid: an endogenous peroxisome proliferator-activated receptor gamma ligand. Proc Natl Acad Sci USA 102, 2340-2345.

Seale, P., Kajimura, S., Yang, W., Chin, S., Rohas, L. M., Uldry, M., Tavernier, G., Langin, D., and Spiegelman, B. M. (2007). Transcriptional control of brown fat determination by PRDM16. Cell Metab 6, 38-54.

Shiu, R. P., and Paterson, J. A. (1984). Alteration of cell shape, adhesion, and lipid accumulation in human breast cancer cells (T-47D) by human prolactin and growth hormone. Cancer Res 44, 1178-1186.

Suzuki, K., Sakata, N., Hara, M., Kitani, A., Harigai, M., Hirose, W., Kawaguchi, Y., Kawagoe, M., and Nakamura, H. (1991). Flow cytometric analysis of lipid droplet formation in cells of the human monocytic cell line, U937. Biochem Cell Biol 69, 571-576.

Terstappen, L. W., Konemann, S., Safford, M., Loken, M. R., Zurlutter, K., Buchner, T., Hiddemann, W., and Wormann, B. (1991). Flow cytometric characterization of acute myeloid leukemia. Part 1. Significance of light scattering properties. Leukemia 5, 315-321.

Thomas, M. E., Morrison, A. R., and Schreiner, G. F. (1995). Metabolic effects of fatty acid-bearing albumin on a proximal tubule cell line. Am J Physiol 268, F1177-1184.

Todaro, G. J., and Green, H. (1963). Quantitative studies of the growth of mouse embryo cells in culture and their development into established lines. J Cell Biol 17, 299-313.

Tontonoz, P., and Spiegelman, B. M. (2008). Fat and beyond: the diverse biology of PPARgamma. Annu Rev Biochem 77, 289-312.

Vierbuchen, T., Ostermeier, A., Pang, Z. P., Kokubu, Y., Sudhof, T. C., and Wernig, M. Direct conversion of fibroblasts to functional neurons by defined factors. (2010). Nature 463, 1035-1041.

Warrell, R. P., Jr., Frankel, S. R., Miller, W. H., Jr., Scheinberg, D. A., Itri, L. M., Hittelman, W. N., Vyas, R., Andreeff, M., Tafuri, A., Jakubowski, A., and et al. (1991). Differentiation therapy of acute promyelocytic leukemia with tretinoin (all-trans-retinoic acid). N Engl J Med 324, 1385-1393.

Waxman, S. (2000). Differentiation therapy in acute myelogenous leukemia (non-APL). Leukemia 14, 491-496.

Yokoi, H., Mizukami, H., Nagatsu, A., Tanabe, H., and Inoue, M. Hydroxy monounsaturated Fatty acids as agonists for peroxisome proliferator-activated receptors. (2010). Biol Pharm Bull 33, 854-861.

We claim:

1. A method for treating a tumor, comprising administering to a subject with a tumor an amount effective of a formulation comprising one or more fatty acids selected from the group consisting of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof, to treat the tumor.

2. The method of claim 1, wherein the method comprises administering two or more of the fatty acids selected from the group.

3. The method of claim 1, wherein the one or more fatty acids consist of palmitoleic acid, oleic acid, and linoleic acid.

4. The method of claim 1, wherein the one or more fatty acids consist of palmitoleic acid, oleic acid, and petroselenic acid.

5. The method of claim 1 wherein the tumor is a solid tumor.

6. The method of claim 1 wherein the tumor is selected from the group consisting of melanoma, hepatic carcinoma, breast carcinoma, and ovarian carcinoma.

7. The method of claim 1, wherein the one or more fatty acids serve to transdifferentiate tumor cells in the tumor into adipocyte-like cells.

8. A pharmaceutical composition comprising
(a) a fatty acid cocktail, wherein fatty acids in the fatty acid cocktail consist of two or more of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof; and
(b) a pharmaceutically accepted carrier.

9. The pharmaceutical composition of claim 8, wherein the fatty acids in the fatty acid cocktail are palmitoleic acid, oleic acid, and linoleic acid.

10. The pharmaceutical composition of claim 8, wherein the fatty acids in the fatty acid cocktail are palmitoleic acid, oleic acid, and petroselenic acid.

11. A method for inducing adipogenesis in an adult non-adipogenic cell, comprising contacting the adult non-adipogenic cell with an amount effective of a formulation comprising one or more fatty acids selected from the group consisting of oleic acid [(9Z)-Octadec-9-enoic acid], linoleic acid [cis, cis-9,12-octadecadienoic acid], palmitoleic acid [hexadec-9-enoic acid], and petroselenic acid [cis-6-octadecenoic], isomers thereof, and pharmaceutically acceptable salts thereof, to induce adipogenesis in the adult non-adipogenic cell.

12. The method of claim 11, wherein the method is carried out in vivo.

13. The method of claim 11, wherein the method is carried out in vitro.
